(19) United States
(12) Patent Application Publication
(10) Pub. No.: US 2006/0240488 A1
(43) Pub. Date: Oct. 26, 2006

(54) Detection of an immune response to GDF-8 modulating agents

(76) Inventors: John A. Nowak, Stratham, NH (US); Denise M. O'Hara, Reading, MA (US); John G. Cryan, Shrewsbury, MA (US); Teresa M. Caiazzo, Tewksbury, MA (US); Alison Joyce, Groveland, MA (US); Joseph W. Rajewski III, South Boston, MA (US); Shujun Sun, Brentwood, NH (US); Neil M. Wolfman, Dover, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/388,030

(22) Filed: Mar. 23, 2006

Related U.S. Application Data
(60) Provisional application No. 60/664,643, filed on Mar. 23, 2005.

(51) Int. Cl. G01N 33/53

(52) U.S. Cl. 435/7.5; 435/7.93

(57) ABSTRACT

This disclosure provides methods for the detection of antibodies to a GDF-8 modulating agent such as, e.g., MYO-029, in a biological sample. Methods to detect an immune response to a GDF-8 modulating agent are also included. In particular, methods to assess an immune response in animals, including humans, to a GDF-8 modulating agent such as a GDF-8 inhibitor are provided herein.

### RELATED CASES

This application claims the benefit of U.S. Provisional Application No. 60/664,643, filed Mar. 23, 2005, the contents of which are incorporated herein in their entirety by reference.

### BACKGROUND

Growth and differentiation factor-8 (GDF-8), also known as myostatin, is a secreted protein and a member of the transforming growth factor-beta (TGF-β) superfamily of structurally related growth factors. Members of this superfamily possess physiologically important growth-regulatory and morphogenetic properties (Kingsley et al., Genes Dev. 8:133-146 (1994); Hoodless et al., Curr. Topics Microbiol. Immunol. 228:235-272 (1998)). Similarly, they share a common structural organization including a short peptide signal for secretion and an amino-terminal portion separated from a bioactive carboxy-terminal portion by a highly conserved proteolytic cleavage site.

Human GDF-8 is synthesized as a 375 amino acid long precursor protein that includes an amino-terminal propeptide portion and a carboxy-terminal mature portion. The propeptide is cleaved from mature GDF-8 at Arg-266. The mature GDF-8 protein is active as a disulfide linked homodimer. Following proteolytic processing, it is believed that two GDF-8 propeptides remain non-covalently complexed with the GDF-8 mature domain dimer, maintaining GDF-8 in a latent, inactive state (Lee et al., Proc. Natl. Acad. Sci. U.S.A. 98:9306-9311 (2001); Thies et al., Growth Factors 18:251-259 (2001)). Other proteins are also known to bind to mature GDF-8 and inhibit its biological activity. Such inhibitory proteins include follistatin and follistatin-related proteins, including GASP-1 (Gamer et al., Dev. Biol. 208:222-232 (1999); U.S. Patent Pub. No. 2003-0180306-A1; U.S. Patent Pub. No. 2003-0162714-A1).

An alignment of deduced amino acid sequences from various species demonstrates that GDF-8 has been highly conserved throughout evolution (McPherron et al., Proc. Nat Acad. Sci. U.S.A. 94:12457-12461 (1997)). In fact, the sequences of human, mouse, rat, porcine, and chicken GDF-8 are 100% identical in the C-terminal region. In baboon, bovine, and ovine GDF-8, the sequences differ by only three amino acids. The zebrafish GDF-8 is more divergent, but it is still 88% identical to human.

GDF-8 is a negative regulator of skeletal muscle mass that is highly expressed in developing and adult skeletal muscle. The GDF-8 null mutation in transgenic mice is characterized by a marked hypertrophy and hyperplasia of the skeletal muscle (McPherron et al., Nature 387:83-90 (1997)). Similar increases in skeletal muscle mass are evident in naturally occurring mutations of GDF-8 in cattle (Ashmore et al., Growth 38:501-507 (1974); Swatland et al., J. Anim. Sci. 38:752-757 (1994); McPherron et al., Proc. Nat. Acad. Sci. U.S.A. 94:12457-12461 (1997); Kambadur et al., Genome Res. 7:910-915 (1997)). Studies have also shown that muscle wasting associated with HIV-infection in humans is accompanied by increases in GDF-8 protein expression (Gonzalez-Cadavid et al., Proc. Natl. Acad. Sci. U.S.A. 95:14938-43 (1998)). In addition, GDF-8 can modulate the production of muscle-specific enzymes (e.g., creatine kinase) and modulate myoblast cell proliferation (WO 00/43781).

Therapeutic agents that inhibit the activity of GDF-8 may be used to treat human or animal disorders in which an increase in muscle tissue would be therapeutically beneficial. Further, it may be desirable to increase muscle mass or muscle strength, or to increase growth or muscle tissue mass in e.g., livestock animals. Thus, there is considerable interest in administering factors that regulate the biological activity of GDF-8 as a pharmaceutical, for example to increase muscle mass, or to treat adipose tissue, muscle, metabolic, and bone-related disorders.

One deleterious side effect encountered in individuals undergoing therapy with, for example, a biological product is an immune response to the therapeutic agent. The administration of a GDF-8 modulating agent to an individual may cause the individual to develop antibodies that specifically bind to the GDF-8 modulating agent. Such an immune response can have serious health consequences. Formation of immune complexes as a result of in vivo administration of a GDF-8 modulating agent may affect the biodistribution and clearance rate of the agent. Such complexes may comprise the administered GDF-8 modulating agent, or a portion thereof, bound to circulating immunoglobulins. In general, formation of immune complexes reduces the amount of therapeutic agent available for therapeutic purposes and may result in retention of the administered agent in non-target tissues. In some cases, circulating immune complexes may accumulate in (and potentially damage) non-target tissues such as the liver and kidneys.

There are a number of GDF-8 modulating agents capable of triggering an immune response in an individual, including inhibitors of GDF-8 activity. MYO-029 is a fully human antibody that is described in further detail in U.S. Patent Pub. No. 2004-0142382. MYO-029 is capable of binding mature GDF-8 with high affinity, inhibiting GDF-8 activity in vitro and in vivo, and inhibiting GDF-8 activity associated with negative regulation of skeletal muscle mass and bone density. MYO-029 promotes increased muscle mass when administered to mice.

Methods to detect an antibody that specifically binds to a GDF-8 modulating agent, such as a biological product, are desirable. In particular, methods allowing the detection and/or quantitation of an immune response to GDF-8 modulating agents, including GDF-8 inhibitors and anti-GDF-8 antibodies are needed. Such methods allow, for example, detecting antibodies to a GDF-8 modulating agent, detecting the presence of an immune response to the agent, monitoring or optimizing the course of therapy, and evaluating candidates for treatment.

### SUMMARY

This invention relates to methods to detect antibodies that specifically bind to a GDF-8 modulating agent in a biological sample. Methods to detect an immune response to a GDF-8 modulating agent are included. In particular, methods to assess an immune response in animals, including humans, to a GDF-8 modulating agent such as a GDF-8 inhibitor are provided herein. In one embodiment, methods to detect the presence of an antibody to a GDF-8 modulating agent such as MYO-029 are provided. In particular, methods to assess the presence and/or quantity of antibodies, including neutralizing antibodies, that specifically bind to a GDF-8 modulating agent in a biological sample from an individual to whom a GDF-8 modulating agent has been administered are provided.

In one embodiment, a method to detect an antibody that specifically binds to a GDF-8 modulating agent in a biological sample is provided, in which the method comprises the steps of: (a) adding the GDF-8 modulating agent to an in vitro assay for a GDF-8 activity in a reaction vessel; (b) adding the biological sample to the in vitro assay for a GDF-8 activity in the reaction vessel; (c) detecting modulation of the GDF-8 activity by the biological sample; and (d) comparing the modulation of the GDF-8 activity in the presence of the biological sample to the modulation of the GDF-8 activity in the presence of the GDF-8 modulating agent alone. In certain embodiments, the in vitro assay is a reporter gene assay. In other embodiments, the in vitro assay is an assay to detect specific binding to the GDF-8 modulating agent, such as an immunoassay, for example.

In certain embodiments, methods to detect an antibody that specifically binds to MYO-029 in a biological sample are provided, comprising the following steps: (a) providing a host cell comprising a reporter gene construct in a reaction vessel, wherein the construct comprises a GDF-8-responsive control element and a reporter gene; (b) adding an amount of mature GDF-8 protein to the vessel sufficient to activate expression of the reporter gene; (c) adding an amount of MYO-029 to the vessel of step (b) sufficient to modulate the GDF-8 activation of the reporter gene; (d) adding a biological sample to the reaction vessel of step (c); and (e) detecting reporter gene expression in the presence and absence of the biological sample.

In one embodiment, methods for the detection of antibodies that specifically bind to a GDF-8 modulating agent in a biological sample are provided, comprising: (a) contacting the GDF-8 modulating agent with a surface of a reaction vessel; (b) adding the biological sample to the reaction vessel; (c) adding a detection agent to the reaction vessel; and (d) detecting a GDF-8 modulating agent/antibody complex associated with the surface of the reaction vessel. In some instances, the detection agent is the GDF-8 modulating agent of step (a) and a detectable label. In some instances, the detection agent is a labeled GDF-8 protein.

In another embodiment, methods to detect an antibody to a GDF-8 inhibitor in a biological sample are provided. The methods comprise: (a) contacting a first GDF-8 inhibitor with a surface of a reaction vessel; (b) adding the biological sample to the reaction vessel; (c) adding a labeled second GDF-8 inhibitor to the reaction vessel; and (d) detecting the labeled GDF-8 inhibitor associated with the surface. In some embodiments, the first GDF-8 inhibitor and the second GDF-8 inhibitor are the same. In some embodiments, the first GDF-8 inhibitor is an antibody that specifically binds to GDF-8. In some embodiments, the second GDF-8 inhibitor is an antibody that specifically binds to GDF-8. In still further embodiments, a GDF-8 inhibitor binds preferentially to GDF-8 over BMP-11.

In a further embodiment, methods to detect an antibody that specifically binds to MYO-029 in a biological sample are provided, comprising: (a) contacting isolated MYO-029 with a surface of a reaction vessel; (b) adding the biological sample to the reaction vessel; (c) adding labeled MYO-029 to the reaction vessel; and (d) detecting labeled MYO-029 associated with the surface.

Methods to detect an antibody that specifically binds to MYO-029 in a biological sample are also provided as a specific embodiment. The methods of this embodiment comprise: (a) contacting isolated MYO-029 with a surface of a reaction vessel; (b) adding the biological sample to the reaction vessel; (c) adding labeled GDF-8 to the reaction vessel; and (d) detecting labeled GDF-8 associated with the surface in the presence and absence of the biological sample.

In another embodiment, methods to assess an individual's immune response to a first GDF-8 inhibitor are provided, comprising: (a) contacting a first GDF-8 inhibitor with a surface of a reaction vessel; (b) adding a biological sample to the reaction vessel; (c) adding a labeled second GDF-8 inhibitor to the reaction vessel; and (d) detecting a labeled second GDF-8 inhibitor/antibody complex associated with the surface, wherein detection of labeled complex indicates an immune response to the first GDF-8 inhibitor.

In a further embodiment, methods to assess an individual's immune response to a first GDF-8 inhibitor are provided. These methods comprise: (a) contacting a GDF-8 inhibitor with a surface of a reaction vessel; (b) adding the biological sample to the reaction vessel; (c) adding a labeled GDF-8 protein to the reaction vessel; and (d) comparing the amount of labeled GDF-8 protein associated with the surface in the test sample to an amount of labeled GDF-8 protein associated with the surface in a control sample, wherein detection of a decreased level of labeled complex in the test sample as compared to the control sample indicates an immune response to the GDF-8 inhibitor.

In particular embodiments, the GDF-8 modulating agent is chosen from an antibody that specifically binds to GDF-8, an antibody that specifically binds to a GDF-8 binding partner, a soluble GDF-8 receptor, an ActRIIB protein, a follistatin-domain containing protein, a follistatin protein, a GASP-1 protein, a GDF-8 protein, a GDF-8 propeptide, a non-proteinacious inhibitor, a nucleic acid, and a small molecule. In some preferred embodiments, the GDF-8 modulating agent is a GDF-8 inhibitor. In some preferred embodiments, the GDF-8 modulating agent is an antibody that specifically binds to GDF-8 such as, e.g., MYO-029.

In certain embodiments, the biological sample is from a mammal, bird, reptile, or fish. In some preferred embodiments, the biological sample is from a human. In particular embodiments the biological sample is chosen from serum, blood, plasma, biopsy sample, tissue sample, cell suspension, saliva, oral fluid, cerebrospinal fluid, amniotic fluid, milk, colostrum, mammary gland secretion, lymph, urine, sweat, lacrimal fluid, gastric fluid, synovial fluid, and mucus. In some preferred embodiments, the biological sample is serum, blood, or plasma.

In various other embodiments, the label is chosen from an enzyme, an epitope tag, a radiolabel, biotin, a dye, a fluorescent tag label, and a luminescent label. In embodiments wherein the label is an enzyme, the methods may further comprise adding a substrate that changes color, luminescence, or fluorescence in the presence of the enzyme. In illustrative embodiments, the label is biotin, and the method further comprises adding an avidin-enzyme conjugate. In one specific embodiment, the method further comprises adding a substrate that changes color, luminescence, or fluorescence in the presence of the enzyme.

Additional aspects and embodiments of the invention will be set forth in part in the description which follows, and in part will be apparent from the description, or may be learned by practice of the invention. This summary and the following description are not intended to be restrictive of the invention, as provided in the claims.

### BRIEF DESCRIPTION OF THE SEQUENCES

DNA and amino acid (AA) sequences of GDF-8, MYO-029, and relevant scFv fragments, V_{H} and V_{L} domains, and complementarity determining regions (CDRs) are set forth in the Sequence Listing and are enumerated as listed in Table 1.

TABLE 1

| | SEQ ID NO |
|---|---|
| AA sequence of mature human GDF-8 | 1 |
| AA sequence of human GDF-8 precursor | 2 |
| DNA sequence of MYO-029 scFv | 3 |
| AA sequence of MYO-029 scFv | 4 |
| DNA sequence of MYO-029 V_{H} | 5 |
| AA sequence of MYO-029 V_{H} | 6 |
| DNA sequence of MYO-029 V_{L} | 7 |
| AA sequence of MYO-029 V_{L} | 8 |
| Germlined DNA seq. of MYO-029 scFv | 9 |
| Germlined AA seq. of MYO-029 scFv | 10 |
| Germlined DNA seq. V_{H} | 11 |
| Germlined AA seq. of MYO-029 V_{H} | 12 |
| Germlined DNA seq. of MYO-029 V_{L} | 13 |
| Germlined AA seq. of MYO-029 V_{L} | 14 |
| AA sequence of MYO-029 H1 | 15 |
| AA sequence of MYO-029 H2 | 16 |
| AA sequence of MYO-029 H3 | 17 |
| AA sequence of MYO-029 L1 | 18 |
| AA sequence of MYO-029 L2 | 19 |
| AA sequence of MYO-029 L3 | 20 |

### BRIEF DESCRIPTION OF THE DRAWINGS

FIG. 1 illustrates one embodiment of the method of the invention, wherein the GDF-8 modulating agent is MYO-029, and the detection agent is biotinylated MYO-029.

FIG. 2 illustrates one embodiment of the method of the invention, wherein the GDF-8 modulating agent is MYO-029 and the detection agent is biotinylated GDF-8.

### DETAILED DESCRIPTION

This invention relates to methods to detect antibodies that specifically bind to a GDF-8 modulating agent in a biological sample. Methods to detect an immune response to a GDF-8 modulating agent are included. In particular, methods to assess an immune response in animals, including humans, to an exogenous GDF-8 modulating agent, such as a GDF-8 inhibitor, are provided herein. In one embodiment, methods to detect the presence of a neutralizing antibody to a GDF-8 modulating agent, for example, MYO-029, are provided. In particular, methods to assess the presence and/or quantity of antibodies that specifically bind to a GDF-8 modulating agent in a biological sample from an individual to whom the GDF-8 modulating agent has been administered are provided.

When a GDF-8 modulating agent is administered to an individual, methods to detect an immune response to the administered GDF-8 modulating agent are useful for determining the presence and/or extent of antibodies that specifically bind to the GDF-8 modulating agent in a biological sample. The methods may also allow one to assess a therapeutic regimen, to track the course of therapy, to assess the suitability of a GDF-8 modulating agent, to identify a candidate for therapy, or to adjust the dosage of the agent, for example. The methods may further allow identification of abuse of a GDF-8 modulating agent.

In order for the present invention to be more readily understood, certain terms are defined herein. Additional definitions are set forth throughout the detailed description.

The term “GDF-8” refers to a specific growth and differentiation factor-8. The term refers to the full-length unprocessed precursor form of GDF-8 as well as the mature and propeptide forms resulting from post-translational cleavage. Unless otherwise specified as “inactive,” a “GDF-8 protein” retains one or more GDF-8 biological activities. The term also refers to any fragments and variants of GDF-8 that maintain at least one biological activity associated with mature GDF-8, as discussed herein, including sequences that have been modified. The amino acid sequence of mature human GDF-8 is provided in SEQ ID NO:1, and the precursor, full-length human GDF-8 sequence is provided in SEQ ID NO:2. The present invention relates to GDF-8 from all vertebrate species, including, but not limited to, human, bovine, chicken, mouse, rat, porcine, ovine, turkey, baboon, and fish (for sequence information, see, e.g., McPherron et al., Proc. Nat. Acad. Sci. U.S.A. 94:12457-12461 (1997)).

The term “mature GDF-8” refers to the protein that is cleaved from the carboxy-terminal domain of the GDF-8 precursor protein. Depending on conditions, the mature GDF-8 may be present as a monomer, homodimer, and/or in a GDF-8 latent complex. In its biologically active form, the mature GDF-8 is also referred to as “active GDF-8.” The term also refers to any fragments and variants of GDF-8 that maintain at least one biological activity associated with mature GDF-8, as discussed herein, including sequences that have been modified.

The term “GDF-8 propeptide” refers to the polypeptide that is cleaved from the amino-terminal domain of the GDF-8 precursor protein. The GDF-8 propeptide is capable of binding to the propeptide binding domain on the mature GDF-8. The GDF-8 propeptide forms a complex with the mature GDF-8 homodimer. It is believed that two GDF-8 propeptides associate with two molecules of mature GDF-8 in the homodimer to form an inactive tetrameric complex, called a latent complex. The latent complex may include other GDF inhibitors in place of or in addition to one or more of the GDF-8 propeptides.

The term “GDF-8 activity” refers to one or more physiologically growth-regulatory or morphogenetic activities associated with active GDF-8 protein. For example, active GDF-8 is a negative regulator of skeletal muscle mass. Active GDF-8 can also modulate the production of muscle-specific enzymes (e.g., creatine kinase), stimulate myoblast proliferation, and modulate preadipocyte differentiation to adipocytes. “GDF-8 activity” includes “GDF-8 binding activity.” For example, mature GDF-8 specifically binds to the propeptide region of GDF-8, to ActRIIB, to a GDF-8 receptor, to activin, to follistatin, to follistatin-domain-containing proteins, to GASP-1, and to other proteins. A GDF-8 inhibitor, such as an antibody or portion thereof, may reduce one or more of these binding activities. Exemplary procedures for measuring GDF-8 activity in vivo and in vitro are set forth below.

The term “GDF-8 modulating agent” includes any agent capable of modulating activity, expression, processing, or secretion of GDF-8, or a pharmaceutically acceptable derivative thereof. Agents that increase one or more GDF-8 activitites and agents that decrease one or more GDF-8 activities are encompassed by the term. The term “GDF-8 inhibitor” includes any agent capable of affecting activity, expression, or processing of GDF-8, or a pharmaceutically acceptable derivative thereof. A GDF-8 inhibitor reduces one or more activities associated with GDF-8. In certain embodiments, a GDF-8 inhibitor will affect binding of GDF-8 to one or more of its physiological binding partners, including, but not limited to a receptor (e.g. ActRIIB), a follistatin-domain containing protein (e.g. follistatin, FLRG, GASP-1, GASP-2), or a GDF-8 protein such as the GDF-8 propeptide and mutants and derivatives thereof. Such GDF-8 inhibitors include, for example, antibodies that specifically bind to GDF-8 (including MYO-029, MYO-028, MYO-022, JA-16, and fragments and derivatives thereof), antibodies that specifically bind to a GDF-8 receptor (see, e.g., U.S. Pat. No. 6,656,475, U.S. Patent Pub. No. 2004/0077053-A1), modified soluble receptors (including receptor fusion proteins, such as the ActRIIB-Fc fusion protein), other proteins that specifically bind to GDF-8 (such as the GDF-8 propeptide, mutants and derivatives of the GDF-8 propeptide, follistatin, follistatin-domain containing proteins, and Fc fusions of these proteins), proteins binding to the GDF-8 receptor and Fc fusions of these proteins, and mimetics are included. Nonproteinaceous inhibitors (such as nucleic acids) are also encompassed by the term GDF-8 inhibitor. GDF-8 inhibitors include proteins, antibodies, peptides, peptidomimetics, ribozymes, anti-sense oligonucleotides, double-stranded RNA, siRNA (e.g. for RNAi), and other small molecules, which specifically inhibit GDF-8. Such inhibitors are said to “inhibit,” “reduce,” or “neutralize” the biological activity of GDF-8, and are described in more detail below.

A “GDF-8 inhibitor” will “inhibit,” “neutralize,” or “reduce” at least one biological activity of GDF-8, such as a physiological, growth-regulatory, or morphogenetic activity associated with active GDF-8 protein. For example, GDF-8 is a negative regulator of skeletal muscle growth. A GDF-8 inhibitor can increase muscle mass, increase muscle strength, modulate the levels of muscle-specific enzymes (e.g., creatine kinase), stimulate myoblast proliferation, and modulate preadipocyte differentiation to adipocytes, decrease fat accumulation, decrease serum triglyceride levels, decrease serum cholesterol levels, modulate glucose metabolism, and reduce hyperglycemia. Also, GDF-8 blocks insulin-induced expression of GLUT4, and it blocks insulin-mediated differentiation of pre-adipocytes.

The terms “inhibit,” “inhibitory,” and their cognates refer to a reduction in one or more activities of GDF-8 by a GDF-8 inhibitor, relative to the activity of GDF-8 in the absence of the same inhibitor. The reduction in activity is preferably at least about 20%, 30%, 40%, 50%, 60%, 70%, 80%, 90%, or higher. In certain embodiments, the activity of GDF-8, when affected by one or more of the presently disclosed inhibitors, is reduced at least 50%, preferably at least 60%, 62%, 64%, 66%, 68%, 70%, 72%, 74%, 76%, 78%, 80%, 82%, 84%, 86%, or 88%, more preferably at least 90%, 92%, 94%, 96%, 98%, or 99%, and even more preferably at least 95% to 100% relative to a GDF-8 protein in the absence of the GDF-8 inhibitor. The terms “neutralize,” “neutralizing,” and their cognates refer to a reduction of one or more GDF-8 activities by at least 80%, 85%, 90%, or 95%. Inhibition of GDF-8 activity can be measured in pGL3(CAGA)₁₂ reporter gene assays (RGA) as described in Thies et al., Growth Factors 18:251-259 (2001) or in ActRIIB receptor assays as illustrated below, for example.

The term “antibody,” as used herein, is any polypeptide comprising an antigen-binding site, such as an immunoglobulin or a fragment thereof, and encompasses any polypeptide comprising an antigen-binding site regardless of the source, species of origin, method of production, and characteristics. As non-limiting examples, the term “antibody” includes synthetic, human, orangutan, monkey, primate, mouse, rat, goat, dog, sheep, and chicken antibodies. The term includes but is not limited to polyclonal, monoclonal, monospecific, polyspecific, non-specific, humanized, single-chain, chimeric, synthetic, recombinant, hybrid, mutated, and CDR-grafted antibodies. For the purposes of the present invention, “antibody” also includes antibody fragments, unless otherwise stated (such as when preceded by the word “intact”). Exemplary antibody fragments include Fab, F(ab′)₂, Fv, scFv, Fd, dAb, and other antibody fragments that retain antigen-binding function. Typically, such fragments comprise an antigen-binding domain. As will be recognized by those of skill in the art, any of such molecules, e.g., a “human” antibody, may be engineered (for example “germlined”) to decrease its immunogenicity, increase its affinity, alter its specificity, or for other purposes.

Antibodies can be made, for example, via traditional hybridoma techniques (Kohler et al., Nature 256:495-499 (1975)), recombinant DNA methods (U.S. Pat. No. 4,816,567), or phage display techniques using antibody libraries (Clackson et al., Nature 352:624-628 (1991); Marks et al., J. Mol. Biol. 222:581-597 (1991)). For various other antibody production techniques, see Antibodies: A Laboratory Manual, eds. Harlow et al., Cold Spring Harbor Laboratory, 1988.

The term “antigen-binding domain” refers to the part of an antibody molecule that comprises the area specifically binding to or complementary to a part or all of an antigen. Where an antigen is large, an antibody may only bind to a particular part of the antigen. The “epitope” or “antigenic determinant” is a portion of an antigen molecule that is involved in specific interactions with the antigen-binding domain of an antibody. An antigen-binding domain may be provided by one or more antibody variable domains (e.g., an Fd antibody fragment consisting of a V_{H} domain). In certain embodiments, an antigen-binding domain comprises an antibody light chain variable region (V_{L}) and an antibody heavy chain variable region (V_{H}) (U.S. Pat. No. 5,565,332).

The terms “specific binding,” “specifically binds,” and the like, mean that two or more molecules form a complex that is measurable under physiologic or assay conditions and is selective. An antibody or other inhibitor is said to “specifically bind” to a protein if, under appropriately selected conditions, such binding is not substantially inhibited, while at the same time non-specific binding is inhibited. Specific binding is characterized by a relatively high affinity and is selective for the compound or protein. Nonspecific binding usually has a low affinity. Typically, the binding is considered specific when the affinity constant Kₐ is at least about 10⁶ M⁻¹, or preferably at least about 10⁷, 10⁸, 10⁹, or 10¹⁰ M⁻¹. Certain methods require high affinity for specific binding, whereas other methods, such as a surface plasmon resonance assay, may detect less stable complexes and lower affinity interactions. If necessary, non-specific binding can be reduced without substantially affecting specific binding by varying the binding conditions. Such conditions are known in the art, and a skilled artisan using routine techniques can select appropriate conditions. The conditions are usually defined in terms of concentration of the binding partners, ionic strength of the solution, temperature, time allowed for binding, concentration of non-related molecules (e.g., serum albumin, milk casein), etc. Exemplary binding conditions are set forth in the Examples.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure.

The term “individual” refers to any vertebrate animal, including a mammal, bird, reptile, amphibian, or fish. The term “mammal” includes any animal classified as such, male or female, including humans, non-human primates, monkeys, dogs, horses, cats, sheep, pigs, goats, cattle, etc. Examples of non-mammalian animals include chicken, turkey, duck, goose, fish, salmon, catfish, bass, frog, and trout. An individual may be chosen from humans, athletes, or domesticated, livestock, zoo, sports, racing, or pet animals, for example.

The term “effective dose,” or “effective amount,” refers to a dosage or level that is sufficient to ameliorate clinical symptoms of, or achieve a desired biological outcome (e.g., increasing muscle mass, muscle strength, and/or bone density) in individuals, including individuals having a GDF-8 associated disorder. Such amount should be sufficient to reduce the activity of GDF-8 associated with negative regulation of skeletal muscle mass and bone density, for example. Therapeutic outcomes and clinical symptoms may include reduction in body fat, increase in muscle mass, improved cardiovascular indicators, or improved glucose metabolism regulation. A GDF-8 inhibitor can increase muscle mass, muscle strength, modulate the levels of muscle-specific enzymes (e.g., creatine kinase), and/or stimulate myoblast proliferation, for example. In a preferred embodiment, a GDF-8 inhibitor reduces clinical manifestations of a GDF-8 associated disorder. A GDF-8 modulating agent can modulate preadipocyte differentiation to adipocytes, decrease fat accumulation, decrease serum triglyceride levels, decrease serum cholesterol levels, modulate glucose metabolism, modulate bone density, and reduce hyperglycemia, for example. A GDF-8 inhibitor may also be administered to an individual in order to increase muscle mass, to improve athletic performance, or to increase or accelerate growth, including muscle growth. The effective amount can be determined as described in the subsequent sections. A “therapeutically effective amount” of a GDF-8 inhibitor refers to an amount which is effective, upon single or multiple dose administration to an individual (such as a human) at treating, preventing, curing, delaying, reducing the severity of, or ameliorating at least one symptom of a disorder or recurring disorder, or prolonging the survival of the subject beyond that expected in the absence of such treatment.

A “GDF-8 associated disorder” is a disorder or condition in which a subject would benefit from the administration of a GDF-8 modulator, such as a GDF-8 inhibitor. A GDF-8 associated disorder includes a medical disorder such as a muscle-related or neuromuscular disorder or condition, or an adipose tissue, metabolic, or bone-related disorder or condition.

Administration of a GDF-8 inhibitor may be “therapeutic” when the inhibitor is administered to an individual to treat a disorder, which includes amelioration and/or prevention of symptoms or of the disorder. Therapeutic uses include the administration of a GDF-8 modulating agent to an individual having a medical disorder or who ultimately may acquire the disorder, in order to prevent, cure, delay, reduce the severity of, or ameliorate one or more symptoms of a disorder or recurring disorder, or in order to prolong the survival of a subject beyond that expected in the absence of such treatment. A GDF-8 inhibitor can increase muscle mass, muscle strength, modulate the levels of muscle-specific enzymes (e.g., creatine kinase), and stimulate myoblast proliferation, for example. A GDF-8 modulating agent can modulate preadipocyte differentiation to adipocytes, decrease fat accumulation, decrease serum triglyceride levels, decrease serum cholesterol levels, modulate glucose metabolism, modulate bone density, and reduce hyperglycemia, for example. A GDF-8 inhibitor may also be administered to an individual in order to increase muscle mass, to improve athletic performance, or to increase or accelerate growth, including muscle growth.

The term “highly stringent” or “high stringency” describes conditions for hybridization and washing used for determining nucleic acid-nucleic acid interactions. Such conditions are known to those skilled in the art and can be found in, for example, Wiley et al., Current Protocols in Molecular Biology, John Wiley & Sons, N.Y., 6.3.1-6.3.6 (1989). Both aqueous and nonaqueous conditions as described in the art can be used. One example of highly stringent hybridization conditions is hybridization in 6× sodium chloride/sodium citrate (SSC) at about 45° C., followed by at least one wash in 0.2×SSC, 0.1% sodium dodecyl sulfate (SDS) at 50° C. Other examples of highly stringent hybridization conditions include hybridization in 6×SSC at about 45° C. (or 50° C., 60° C., or 65° C.) followed by at least one wash in 0.2×SSC, 0.1% SDS at about 55° C., 60° C. or 65° C. Highly stringent conditions may also be hybridization in 0.5M sodium phosphate, 7% SDS at 65° C., followed by at least one wash at 0.2×SSC, 1% SDS at 65° C. (see also, e.g., Sambrook et al., Molecular Cloning A Laboratory Manual, Cold Spring Harbor Laboratory Press, 1989).

The phrase “substantially identical” or “substantially similar” means that the relevant amino acid or nucleotide sequence, such as of the GDF-8 inhibitors of the invention, will be identical to or have insubstantial differences (through conserved amino acid substitutions) in comparison to the sequences which are disclosed. Nucleotide and polypeptides of the invention include, for example, those that are at least about 60%, 65%, 70%, 75%, 80%, 85%, 90%, 92%, 93%, 94%, 95%, 96%, 97%, 98%, or 99% identical in sequence to nucleic acid molecules and polypeptides disclosed.

For polypeptides, at least 20, 30, 50, 100, or more amino acids will be compared between the original polypeptide and the variant polypeptide that is substantially identical to the original. For nucleic acids, at least 50, 100, 150, 300 or more nucleotides will be compared between the original nucleic acid and the variant nucleic acid that is substantially identical to the original. Thus, a variant could be substantially identical in a region or regions, but divergent in others, while still meeting the definition of “substantially identical.” Percent identity between two sequences is determined by standard alignment algorithms such as, for example, Basic Local Alignment Tool (BLAST) described in Altschul et al., J. Mol. Biol., 215:403-410 (1990), the algorithm of Needleman et al., J. Mol. Biol., 48:444-453 (1970), or the algorithm of Meyers et al., Comput. Appl. Biosci., 4:11-17 (1988).

The term “variant” refers to nucleotide and amino acid sequences that are substantially identical or similar to the nucleotide and amino acid sequences of, for example, the GDF-8 inhibitors provided, respectively. Variants can be naturally occurring, for example, naturally occurring human and non-human nucleotide sequences, or be generated artificially. Examples of variants are those resulting from alternative splicing of the mRNA, including both 3′ and 5′ spliced variants, point mutations and other mutations, or proteolytic cleavage of the proteins. Variants include nucleic acid molecules or fragments thereof and amino acid sequences and fragments thereof, that are substantially identical or similar to other nucleic acids (or their complementary strands when they are optimally aligned (with appropriate insertions or deletions) or amino acid sequences respectively. In one embodiment, there is at least about 60%, 65%, 70%, 75%, 80%, 85%, 90%, 92%, 93%, 94%, 95%, 96%, 97%, 98%, or 99% identity between a nucleic acid molecule or protein of the invention and another nucleic acid molecule or protein respectively, when optimally aligned. Additionally, variants include proteins or polypeptides that exhibit GDF-8 activity or inhibit GDF-8 activity, as discussed in this application.

A “biological sample” is biological material collected from an individual, such as cells, tissues, organs, or fluids. Exemplary biological samples include serum, blood, and plasma.

The term “reaction vessel” refers to a container in which an in vitro assay such as an association reaction between a GDF-8 modulating agent and an antibody can occur and be detected. A “surface” is the outer part of any solid (such as, e.g., glass, cellulose, polyacrylamide, nylon, polystyrene, polyvinyl chloride, dextran sulfate, or treated polypropylene) to which a GDF-8 modulating agent can be directly or indirectly “contacted,” “immobilized,” or “coated.” A “surface of a reaction vessel” may be a part of the vessel itself, or the surface may be in the reaction vessel. A surface such as polystyrene, for example, may be subjected to chemical or radiation treatment to change the binding properties of its surface. Medium binding, high binding, aminated, and activated surfaces are encompassed by the term. A GDF-8 modulating agent can be directly contacted with a surface, e.g., by physical adsorption or covalent binding to the surface, or it can be indirectly contacted, e.g., through an interaction with a substance or moiety that is directly contacted with the surface.

The term “capture agent” as used herein, refers to the molecule, such as a protein, for example, that is used in an immunoassay to specifically bind to a target protein, such as a GDF-8 modulating agent or GDF-8 itself. A capture agent suitable for the instant methods specifically binds to the GDF-8 modulating agent and/or to GDF-8 protein. For example, a capture agent may be GDF-8 protein, including a mature GDF-8 dimer, or a protein that specifically binds to a GDF-8 protein. Similarly, a capture agent may be a GDF-8 modulating agent or a protein that specifically binds to a GDF-8 modulating agent.

A “detection agent” is a protein or small molecule that specifically binds to an antibody to a GDF-8 modulating agent. A detection agent may optionally comprise a detectable label. A detection agent may also be itself detected by a detection agent comprising a detectable label. The term “label” refers to a molecule which, by its chemical nature, provides an analytically identifiable signal which allows the detection of a molecular interaction. A protein, including an antibody, has a detectable label if it is covalently or non-covalently bound to a molecule that can be detected directly (e.g., by means of a chromophore, fluorophore, or radioisotope) or indirectly (e.g., by means of catalyzing a reaction producing a colored, luminescent, or fluorescent product).

The present invention relates to methods to detect an immune response to a GDF-8 modulating agent as well as methods to detect antibodies to a GDF-8 modulating agent in a biological sample. In one embodiment, the methods detect antibodies, in particular antibodies capable of binding to constituents of in vivo therapeutic GDF-8 modulating agents, including GDF-8 inhibitors. In some embodiments, the assays detect neutralizing antibodies, such as antibodies that inhibit the action of the GDF-8 modulating agent. In a particular embodiment, the assays detect antibodies that inhibit the binding of MYO-029 to GDF-8. The methods are useful in evaluating the suitability of human patients to receive therapeutic antibodies, or other GDF-8 modulating agents, for example, that inhibit a biological activity of GDF-8. In a specific embodiment, the methods detect the presence of antibodies in a biological sample to MYO-029.

An individual with a GDF-8 associated disorder, an individual at risk for developing a GDF-8 associated disorder, an individual undergoing therapy with a GDF-8 modulating agent, and an individual who is a candidate for administration of a GDF-8 modulating agent, may be a candidate for the methods herein provided. The methods of the invention may detect or prevent a deleterious immune response, and/or assess efficacy, biological stability, or suitability of use of a GDF-8 modulating agent.

An individual having; or at risk for developing, a GDF-8 associated disorder such as a muscle-related disorder or a neuromuscular disorder is a candidate for the methods provided herein. Inhibition of GDF-8 activity increases muscle tissue in individuals, including those suffering from muscle-related disorders. A number of disorders are associated with functionally impaired muscle or nerve tissue, e.g., muscular dystrophies, amyotrophic lateral sclerosis (ALS), sarcopenia, cachexia, muscle wasting, muscle atrophy, or muscle degeneration, including wasting, atrophy, or frailty. Muscular dystrophies include, for example, pseudohypertrophic, facioscapulohumeral, and limb-girdle muscular dystrophies. Exemplary muscular dystrophies include Duchenne's muscular dystrophy (Leyden-Möbius), Becker muscular dystrophy, Emery Dreifuss muscular dystrophy, limb girdle muscular dystrophy, rigid spine syndrome, Ullrich syndrome, Fukuyama muscular dystrophy, Walker Warburg syndrome, muscle eye brain disease, facioscapulohumeral muscular dystrophy (Landouzy-Dejerine), congenital muscular dystrophy, myotonic dystrophy (Steinert's disease), myotonia congenital, and Gowers disease.

A GDF-8 associated muscle disorder also includes a disorder chosen from muscle degeneration associated with cardiovascular disease, or secondary to another disease or condition such as organ atrophy, organ failure, cancer, Acquired Immune Deficiency Syndrome (AIDS), bed rest, immobilization, prolonged lack of use, or other disease or condition are also included in the term.

An individual having, or at risk for developing, adipose tissue disorders (e.g., obesity), cardiovascular disorders (when associated with muscle loss or muscle wasting), and disorders of insulin metabolism may be a candidate. Similarly, individuals having, or at risk for developing, a disorder associated with a loss of bone, including osteoporosis, especially in the elderly and/or postmenopausal women, glucocorticoid-induced osteoporosis, osteopenia, osteoarthritis, and osteoporosis-related fractures are candidates for the methods herein. Other GDF-8 associated conditions include metabolic bone diseases and disorders characterized by low bone mass, such as those due to chronic glucocorticoid therapy, premature gonadal failure, androgen suppression, vitamin D deficiency, secondary hyperparathyroidism, nutritional deficiencies, and anorexia nervosa.

Examples of cardiovascular disorders include coronary artery disease (atherosclerosis), angina (including acute angina and unstable angina), heart attack, stroke (including ischemic stroke), hypertension associated cardiovascular diseases, heart failure, congestive heart failure, coronary artery disease, hypertension, hyperlipidemia, peripheral arterial disease, and peripheral vascular disease. Examples of disorders of insulin metabolism include conditions associated with aberrant glucose homeostasis, type 2 diabetes, prediabetes, impaired glucose tolerance, dyslipidemia, metabolic syndrome (e.g., syndome X), and insulin resistance induced by trauma such as burns or nitrogen imbalance.

Further, an individual desiring to increase muscle mass or muscle strength, for example to improve athletic performance or to increase growth or muscle tissue mass in livestock animals, is a candidate for a method provided herein. An individual exhibiting an increase in muscle mass, such as an increase in muscle cell size (hypertrophy) or muscle cell number (hyperplasia) may be a candidate for a method to detect an antibody to an exogenous GDF-8 modulating agent. The increase can be in type 1 and/or type 2 muscle fibers of a mammal or other animal. Methods to measure an increase in muscle mass are well known in the art. For example, muscle can be measured before and after administration of a GDF-8 modulating agent using standard techniques such as underwater weighing. An increase in muscle size may be evidenced by weight gain of at least about 5%, 10%, 20%, or more.

In one embodiment, the present invention comprises a method to detect an antibody that specifically binds to a GDF-8 modulating agent in a biological sample from at least one individual, which comprises the steps of: (a) adding the GDF-8 modulating agent to an in vitro assay for a GDF-8 activity in a reaction vessel; (b) adding the biological sample to the in vitro assay for a GDF-8 activity in the reaction vessel; (c) detecting modulation of the GDF-8 activity by the biological sample; and (d) comparing the modulation of the GDF-8 activity in the presence of the biological sample to the modulation of the GDF-8 activity in the presence of the GDF-8 modulating agent alone. In certain embodiments, the in vitro assay is an immunoassay to detect binding of the antibody to the GDF-8 agent, for example, in an enzyme-linked immunosorbent assay (ELISA) format. In one embodiment, the binding to the GDF-8 agent is detected with a detection agent that is the GDF-8 modulating agent with a detectable label. In another embodiment, the detection agent is a labeled GDF-8 protein. In another embodiment, the in vitro assay is a cell-based assay for GDF-8 activity such as, for example, a reporter gene assay.

In certain embodiments, the in vitro assay measures one or more physiologically growth-regulatory or morphogenetic activities associated with active GDF-8 protein. In vitro assays to detect modulation of a GDF-8 activity may be chosen from a cell-based assay or cell-free assay (such as, e.g., an assay to measure modulation of transcription, replication or cell cycle arrest) or a binding assay (such as, e.g., an immunoassay, a surface plasmon resonance assay, immunoprecipitation, or a radioimmune assay). For example, active GDF-8 is a negative regulator of skeletal muscle mass, it modulates the production of muscle-specific enzymes (e.g., creatine kinase), stimulates myoblast proliferation, and modulates preadipocyte differentiation to adipocytes. In some methods, selection of GDF-8 modulating agents from BMP-11 modulating agents is performed. Cell-based and cell free assays for a GDF-8 activity are known in the art and are described infra.

Binding Assays

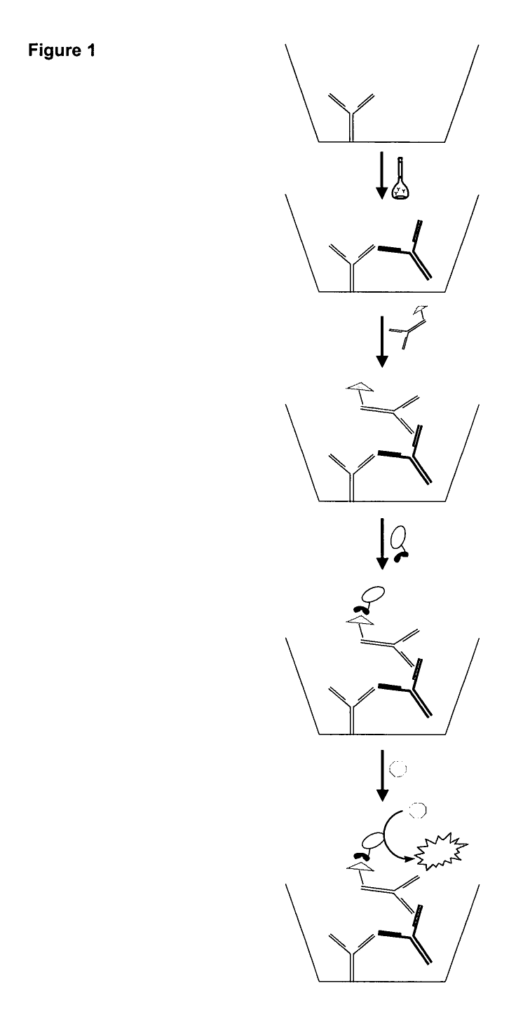

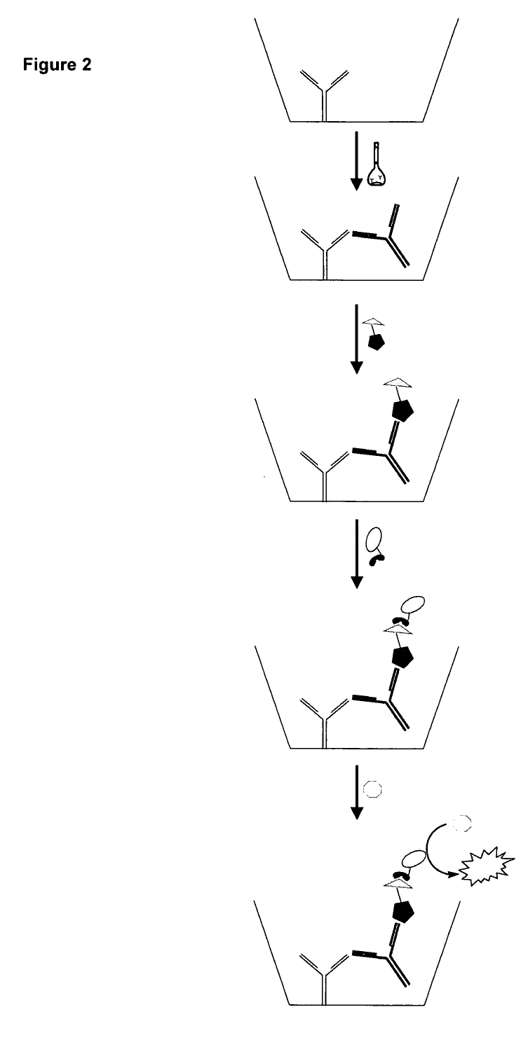

In one embodiment, the present invention comprises a method for detecting the presence of an antibody in a biological sample selected from one or more patient samples, which comprises the following steps: (a) contacting the GDF-8 modulating agent with a surface of a reaction vessel; (b) adding the biological sample to the reaction vessel; (c) adding a detection agent to the reaction vessel; and (d) detecting a GDF-8 modulating agent/antibody complex associated with the surface of the reaction vessel. Two embodiments are depicted in FIGS. 1 and 2.

In step (a) of certain embodiments, the GDF-8 modulating agent is contacted with the surface of a reaction vessel, for example by being either covalently or non-covalently bound to the surface. The contact may be direct or indirect. The solid surface is typically glass or a polymer, such as, e.g., cellulose, dextran sulfate, polyacrylamide, nylon, polystyrene, polyvinyl chloride or polypropylene and may be in the form of a bead, including a magnetic or paramagnetic bead. The surface may be modified, for example by chemical or radiation treatment to affect the binding characteristics of the surface. Immobilization of the ligands on the surface can be achieved by covalent or non-covalent interactions, such as physical adsorption. A GDF-8 modulating agent, for example, MYO-029, may be adsorbed directly to the surface of a reaction vessel. In other embodiments, a GDF-8 modulating agent may be associated with a reaction vessel surface via the interaction of a biotin molecule, covalently bonded to the agent, with an avidin molecule, contacted with the surface of the reaction vessel. Covalent bonding methods include coupling with a crosslinking agent such as glutaraldehyde, hexamethylene isocyanate, a sulfo-containing agent, a peptide, an alkylating agent, or a similar reagent. In some preferred embodiments, the GDF-8 modulating agent is an antibody that specifically binds to GDF-8, a monoclonal antibody that specifically binds to GDF-8, a neutralizing antibody to GDF-8, MYO-029, MYO-028, MYO-022, or JA-16, or a fragment of any of the same. Structural and functional characteristics of these GDF-8 inhibitors are set forth, for example in U.S. Patent Pub. Nos. 2004/0142382-A1 and 2003/0138422-A1, and those portions are specifically incorporated herein by reference, in addition to incorporation of the entire documents. In particular, characteristics of certain neutralizing antibodies, including MYO-029, are described in U.S. Patent Pub. No. 2004/0142382-A1 in paragraphs 54-90, and claims 1-42. Similarly, antibody inhibitors of U.S. Patent Pub. No. 2003/0138422-A1, are described in paragraphs 56-70, 93-110, and claims 1-54.

In certain embodiments, after contacting the GDF-8 modulating agent with the surface of the reaction vessel, the reaction vessel is washed to remove unattached GDF-8 modulating agent prior to addition of the biological sample. Non-specific interactions are minimized with a blocking step, wherein a buffer comprising at least one blocking agent, such as a protein that does not specifically bind to the target is added to the reaction vessel. In other embodiments, detergents may be added, such as ionic or non-ionic detergents. Blocking buffers may comprise serum, bovine serum albumin, milk, casein, gelatin, and/or non-ionic detergents, for example. In some embodiments the reaction vessel is washed with a buffer with between about pH 5 and about pH 9, such as citrate buffer, phosphate buffer, Tris buffer or acetate buffer.

Certain embodiments comprise step (b), in which a biological sample is added to the reaction vessel. The biological sample to be tested may be chosen from serum, blood, plasma, biopsy sample, tissue sample, cell suspension, saliva, oral fluid, cerebrospinal fluid, amniotic fluid, milk, colostrum, mammary gland secretion, lymph, urine, sweat, and lacrimal fluid. In preferred embodiments, the biological sample is a fluid. In some preferred embodiments, the biological sample is chosen from blood, serum, and plasma. In specific embodiments, the biological sample is serum, such as human, monkey, rat, or mouse serum.

In other embodiments, the biological sample is isolated from an individual or individuals and optionally treated prior to testing. The biological sample may be used as collected or after dilution with a suitable diluent. Dilutions are optimized to reduce and/or eliminate matrix interference with the assay. The diluent is not particularly restricted but may comprise deionized water or various buffers having a buffer action within the range of about pH 5 to about pH 9, preferably about pH 6.5 to about pH 8.5, (e.g. citrate buffer, phosphate buffer, Tris buffer, acetate buffer, or borate buffer). In some preferred embodiments, the diluent comprises normal human serum. The diluent may comprise a constant concentration of a control biological sample, chosen to correspond to the test biological sample, for example to control for background effects or interference of the sample matrix.

In one embodiment, a test sample of human serum is diluted in THST (50 mM Tris-HCl, pH 8.0, containing 1.0 mM glycine, 0.5 M NaCl, and 0.05% (v/v) Tween 20®) buffer 1:8 fold, and dilutions of the test sample beyond 8-fold are prepared in THST plus 12.5% human serum. A sample may be diluted approximately 2, 4, 8, 16, 32, 64, or 128-fold. In other embodiments, a test sample is serially diluted 1:1.5 or 1:1.6 to obtain a range of data points that allow verification of dilutional linearity and matrix effects. For preferred biological sample matrices, a dilution may be selected at which matrix interference and assay sensitivity are optimized.

In some embodiments, the sample may be optionally fractionated or concentrated using well known methods and then added to a method provided herein to detect a GDF-8 modulating agent. Fractionation (including purification) or concentration may be used, for example, if matrix interference limits detection of a GDF-8 modulating agent in the assay. Fractionation and concentration techniques, include, but are not limited to, centrifugation, ammonium sulfate precipitation, polyethylene glycol precipitation, trichloroacetic acid (TCA) precipitation, affinity techniques (such as immunoprecipitation with a resin conjugated to a specific binding partner such as an antibody, i.e., an anti-human Fc antibody, protein A or protein G, for example), chromatographic techniques, and other separation techniques. In preferred embodiments, the biological sample is not fractionated or concentrated prior to detection of a GDF-8 modulating agent.

A biological sample may be collected from a naïve individual, or a sample may be taken before, during or after administration of a GDF-8 modulating agent. For example a sample may be obtained from an individual 1, 2, 4, 6, 8, 10, 12, 15, 20, 25, 30, or more days after administration of a GDF-8 modulating agent. A sample may also be obtained 1, 2, 3, 4, 6, 8, 10, 12, 16, or more weeks after administration of a GDF-8 modulating agent. As substantial quantities of circulating GDF-8 modulating agent may compromise detection of antibodies to the agent in certain embodiments of the methods provided herein (see, e.g., Example 7), the timing of sample collection may be optimized to reduce interference from a GDF-8 modulating agent. The persistence of an antibody response is also tested by examining extended timepoints. In some cases, timepoints of up to a year or beyond are appropriate.

In certain embodiments, an aliquot of the sample to be tested is contacted with the immobilized antigen and incubated for a period of time sufficient (e.g., 2-120 minutes, 1-4 hours) and under suitable conditions (e.g., 23° C.) to allow binding of any antibody to the GDF-8 modulating agent present in the sample and to allow antibody/GDF-8 modulating agent complex to form. In other embodiments, the GDF-8 modulating agent/antibody reaction is not particularly restricted but can be conducted under the conditions in routine use for conventional immunoassays. A typical procedure comprises incubating or allowing a reaction system to stand comprising the antibody and GDF-8 modulating agent generally at a temperature of not over 45° C., preferably between about 4° C. and about 40° C., more preferably between about 23° C. and about 40° C. for between about 0.5 and 40 hours, preferably between about 1 and about 20 hours. In preferred embodiments, the reaction buffer is selected to avoid interfering with the reaction or the detection thereof. Therefore, embodiments include, but are not limited to, buffers at between about pH 5 and about pH 9, such as citrate buffer, phosphate buffer, Tris buffer, and acetate buffer.

In certain embodiments, step (c) comprises adding a detection agent to the reaction vessel. Following the incubation period, the immobilized antibody to GDF-8 modulating agent is, in some embodiments, washed with buffer to remove unbound solutes before step (c). In other embodiments a simultaneous assay is performed, whereby steps (b) and (c) occur concurrently.

In particular embodiments, in which step (c) is conducted after step (b), a procedure may comprise incubating or allowing to stand a reaction system comprising the antibody and detection agent generally at a temperature of not over 45° C., preferably between about 4° C. and about 40° C., more preferably between about 25° C. and about 40° C. for between about 0.5 and 40 hours, preferably between about 1 and about 20 hours. In certain embodiments, the reaction buffer is selected so that it does not interfere with the reaction or the detection thereof. Therefore, embodiments include, but are not limited to, buffers at between about pH 5 and about pH 9, such as citrate buffer, phosphate buffer, Tris buffer, and acetate buffer.

In certain embodiments, the detection agent is a molecule that can specifically bind to an antibody that specifically binds to a GDF-8 modulating agent. In some embodiments, the detection agent comprises a detectable label. Preferred detection agents include certain immunoglobulins, and reagents capable of binding to human immunoglobulin sequences (including goat anti-human antibodies, protein A, protein G, etc.), e.g., a constant portion of the immunoglobulin. Immunoglobulins that specifically bind to a GDF-8 modulating agent are included. As MYO-029 is a human IgG1 with a lambda light chain, in various embodiments, detection agents will include reagents capable of binding to human immunoglobulins with lambda light chains. In other embodiments an agent that binds to a non-human IgG1 immunoglobulin with lambda light chains is included. In various embodiments, the detection is qualitative or it is quantitative. In some embodiments, the label will be detectable by visual means without the aid of instruments.

In a preferred embodiment, the detection agent such as MYO-029 or mature GDF-8 dimer is biotinylated. Functional, mature GDF-8 protein, for example, may be biotinylated with amine-specific reagents as set forth in Example 12. Similarly, in an alternative preparation, GDF-8 protein in the latent complex is produced and isolated according to the assay of Example 1 of U.S. Patent Pub. No. 2004/0142382 A1. The latent complex is subsequently biotinylated using well known techniques and/or as described herein.

Mature GDF-8 is unexpectedly sensitive to biotinylation of primary amine groups, such as on lysine residues. Hyperbiotinylated GDF-8, when biotinylated with amine specific biotinylation reagents, is less active or inactivated as compared to GDF-8 without biotin. To retain functional, mature GDF-8 protein after biotinylation, the amount of biotin incorporated into the mature GDF-8 preparation on amine groups was found to be critical. For example, MYO-029 and ActRIIB binding activities are reduced in hyperbiotinylated preparations. Therefore, amine biotinylated mature GDF-8 preparations having less than five moles of biotin per mole of GDF-8 dimer are preferred. In alternate embodiments, proteins may be biotinylated on sulfhydryls, carboxyls, and/or carbohydrates. Photoreactive biotin compounds that non-specifically bind or react upon photoactivation are also available.

In certain methods provided herein, GDF-8 is biotinylated with an amine-specific biotinylation reagent as a latent complex, and subsequently mature GDF-8 is isolated from the complex. In these methods, the amount of biotin incorporated into the mature GDF-8 dimer is optimized to retain biological activity, for example to avoid inactivating the receptor binding site. GDF-8 protein may also be biotinylated on surface cysteine residues (or surface thiol groups) using a sulfhydryl-specific biotinylation reagent. Additionally, methods to biotinylate carbohydrates involving oxidative pretreatment to generate reactive aldehydes and the use of biotin hydrazide reagents, for example, are known in the art and may be optimized for proteins described herein, including for mature GDF-8 protein, optimally in modified form. Further, carboxyl reactive biotinylation reagents and reactions that allow biotinylation via aspartate and glutamate residues, for example, may be used. As would be apparent to one of skill in the art, the optimal molar ratios of biotin to GDF-8 dimer will vary with the biotinylation procedure and reagent utilized. For example, a skilled artisan will appreciate how to optimize an active biotinylated GDF-8 preparation using the methods described herein in combination with known biotinylation procedures, to produce a biotinylated mature GDF-8 protein that has different optimal molar ratios of biotin to GDF-8 dimer, while retaining at least one GDF-8 activity.

Various biotinylation reagents are capable of efficient labeling of proteins, including a GDF-8 latent complex. Molar ratios of biotin derivative to GDF-8 latent complex in the reaction may be about 10, 15, 20, 40, or 80 to 1, and reagent composition and concentration reaction times, and temperatures may be varied to adjust the amount of biotin incorporated in the reaction. For example, salts and other agents may optionally be optimized. In an embodiment, the mature GDF-8 dimer is biotinylated in association with the amino terminal propeptide portion of GDF-8 to avoid inactivating the mature dimer during the biotinylation reaction. Biotin derivatives are well known and available in the art. Modifications of biotin include variable spacer arms, modifications to affect solubility, and/or reactive groups, for example, to allow cleavage of the biotin moiety. Succinimidyl esters of biotin and its derivatives, including water soluble sulfosuccinimidyl esters may be used for biotinylation of GDF-8 on lysine residues, for example. To quantitate the amount of biotin incorporated, for example, well known analytical and sizing techniques are used, including reverse phase high pressure liquid chromatography, mass spectroscopy, etc. Additionally, commercial kits for quantitating biotin by colorimetric or fluorimetric assays, for example, are available (see, e.g., EZ™ Biotin Quantitation Kit, Pierce, utilizing HABA (2-(4′-hydroxyazo benzene)-benzoic acid)).

A further exemplary biotinylation procedure, for example, includes biotinylating GDF-8 latent complex at a ratio of ratio of 15 or 20 moles of EZ-link Sulfo-NHS-Biotin (Pierce) to 1 mole of the GDF-8 complex for 2 hours at 2-8° C. (see, for example, Example 3 of U.S. Patent Pub. No. 2004/0142382 A1). The reaction may be terminated by dropping the pH using 0.5% TFA and then the complex is subjected to chromatography on a C4 Jupiter 250×4.6 mm column (Phenomenex) to separate mature GDF-8 from GDF-8 propeptide. Biotinylated mature GDF-8 fractions eluted with a TFA/CH3CN gradient are pooled, concentrated and quantified by MicroBCA™ protein Assay Reagent Kit (Pierce), or using other well known isolation and concentration techniques.

In a preferred embodiment, an in vitro binding assay comprises a biotinylated GDF-8 protein capture agent, and the GDF-8 protein contacts the surface of the reaction vessel through interaction of the biotin moiety with avidin on the surface of the reaction vessel. In some embodiments, the molar ratio of biotin moiety to mature GDF-8 protein is between about 0.5:1 and about 4:1 in the biotinylated mature GDF-8 protein. In other embodiments, the mean ratio of biotin to GDF-8 dimer is less than about 5 to 1, less than about 2 to 1, or less than about 1 to 1. The ratio of biotin to mature GDF-8 protein has been measured to be a mixture of molar ratios of 0 to 3 in active GDF-8 preparations, with the majority of the molecules being at about 1:1. In some embodiments, the biotinylated mature GDF-8 preparation includes less than about 1, 2, 3, 4, or 5 moles of biotin per mole of mature GDF-8 dimer. The mean or median ratio of biotin to mature GDF-8 protein may be less than or approximately, 0.5, 0.75, 1, 1.25, 1.5, 2, 2.5, 3, 3.5, 4, 4.5, 5, 5.5, 6, 7, 8, or 9, for example. The mode for the ratio of biotin to mature GDF-8 protein may be less than or approximately 1, 2, 3, 4, or 5, for example. Other detection and capture agents may also be labeled by biotinylation. For example, biotinylated MYO-029 may be biotinylated up to a ratio of at least (or less than) 10:1, 20:1, or higher, for example. In preferred embodiments, the mean molar ratio of a biotinylated mature GDF-8 protein preparation is between approximately 1 and 3 moles of biotin to 1 mole of mature GDF-8 dimer. Optionally, another capture agent may be used.

In one embodiment, biotinylated MYO-029 is a detection agent for detecting anti-MYO-029 antibodies binding to MYO-029 immobilized on a surface of a reaction vessel, such as a 96 well plate. In a similar manner, ELISAs to detect antibodies to follistatin, various GDF-8 binding receptors, activin, or GDF-8 propeptide are encompassed within these methods by substituting these materials and their respective biotinylated versions for MYO-029 and biotinylated MYO-029. In addition, any reagent that can recognize and bind to antibodies formed against an inhibitor of GDF-8, whether used alone or in combination with other reagents to generate a practicable dose-response signal, may be utilized to detect antibodies to inhibitors of GDF-8. These reagents could be used in a direct-binding assay format (especially for samples of non-human origin) or in a competitive format (described below).

In further embodiments, the detection agent is complexed by specific binding to an antibody that is also complexed by specific binding to a GDF-8 modulating agent (“bridging”). This bridging assay is made possible by the multivalency of the analyte antibody. In certain embodiments, the presence or absence of the target antibody in a sample or its content is evaluated by measuring the label activity, which depends on the labeling agent used in the labeling of the detection agent.

In some embodiments, a “direct” label may be any molecule bound or conjugated to a specific binding member which is capable of spontaneously producing a detectable signal without the addition of ancillary reagents. Some examples include a radioisotope (e.g., ¹²⁵I, ³H, ¹⁴C), a heavy metal, a fluorophore (e.g., luciferase, green fluorescent protein, fluorescein isothiocyanate, tetramethylrhodamine isothiocyanate, 1-N-(2,2,6,6-tetramethyl-1-oxyl-4-piperidyl)-5-N-(aspartate)-2,4-dinitrobenzene), a dye (e.g., phycocyanin, phycoerythrin, Texas Red, o-phthalaldehyde), luminescent molecules, including chemiluminescent and bioluminescent molecules, colloidal gold particles, colloidal silver particles, other colloidal metal particles, Europium, polystyrene dye particles, minute colored particles such as dye sols, and colored latex particles. Many such substances are well known to those skilled in the art.

In certain cases, the label may be an enzyme such as, e.g., alkaline phosphatase, peroxidase (e.g., horseradish peroxidase), glucose oxidase, or β-galactosidase. In various embodiments, the substrates to be used with the specific enzymes are chosen for the production, in the presence of the corresponding enzyme, of a detectable change in color, fluorescence, or luminescence. The enzyme may be conjugated to the GDF-8 modulating agent by glutaraldehyde or reductive amination cross-linking. As will be readily recognized, however, a wide variety of different conjugation techniques exist and are readily available to the skilled artisan.

In a particular embodiment, the biotinylated and/or enzyme-labeled detection agent such as an antibody is added to the GDF-8 modulating agent/antibody complex, and allowed to bind. The excess reagent is washed away, and a solution containing an appropriate substrate is then added to the reaction vessel. The substrate undergoes an enzyme-catalyzed reaction resulting in a spectrophotometrically-measurable change that is indicative of the amount of antibody present in the sample.

Peroxidase, when incubated with soluble substrates (e.g., 3,3′,5,5′ tetramethylbenzidine (TMB), o-phenylene diamine (OPD), 2,2′-azino-di [3-ethyl-benzthiazoline] sulfonate (ABTS), para nitrophenyl phosphate, luminol, polyphenols, acridine esters, and luciferin), results in a chromogenic or luminescent change in the substrate that can be detected spectroscopically. Typically, after a fixed incubation period with the substrate, the reaction is quenched (e.g., by acidification), and the result is quantified by measuring optical density (absorbance) or luminescence. Absorbance results can be compared with the OD values in the linear range for chomogenic reactions, and luminescent immunoassays are measured in relative light units (RLU). As a further alternative, any combination of reagents that results in binding and the generation of a practicable dose-response signal may be used (e.g., radiolabelled agents, enzyme/substrate reagents, or detection amplification systems utilizing biotin/avidin, for example).

In yet other embodiments, the label is biotin, a hapten, or an epitope tag (e.g., histidine-tag, HA-tag (hemagglutinin peptide), maltose binding protein, AviTag®, or glutathione-S-transferase), which can be detected by the addition of a labeled detection agent that interacts with the label associated with the GDF-8 modulating agent complex. A biotin-labeled (“biotinylated”) detection agent may be detected through its interaction with an avidin-enzyme, e.g., avidin-horseradish peroxidase, conjugate after sequential incubation with the avidin-enzyme conjugate and a suitable chromogenic or fluorogenic substrate. A biotinylated GDF-8 modulating agent may also be detected with Europium labeled streptavidin, in particular embodiments.

In step (d) of certain embodiments, a GDF-8 modulating agent/antibody complex associated with the surface of the reaction vessel is detected by qualitative or quantitative assessment of the signal of the label. In some instances, the label is measured directly, e.g., by fluorescence or luminescence, or indirectly, via addition of a substrate. In others, the label is measured following incubation with an additional reagent. In embodiments in which the label is biotin, an avidin conjugate (such as horseradish peroxidase in some preferred embodiments) may be added in a subsequent step. In one particular embodiment, the avidin conjugate may bind to the immobilized detection agent. Excess avidin conjugate is washed away. A substrate of the enzyme is then added, resulting in a measurable change in, e.g., color, fluorescence, or luminescence. In some embodiments the substrate for horseradish peroxidase is 3,3′,5,5′-tetramethylbenzidine.

The detection agent in steps (c) and (d) is, in some embodiments (e.g., the embodiment depicted in FIG. 1), a second, labeled GDF-8 modulating agent. The GDF-8 modulating agent can be an antibody, including an antibody that specifically binds to GDF-8, an antibody that specifically binds to a GDF-8 binding partner, a GDF-8 receptor, an ActRIIB protein, a follistatin-domain containing protein, a follistatin protein, a GASP-1 protein, a GDF-8 protein, a GDF-8 propeptide, a non-proteinacious inhibitor, and a small molecule. In some embodiments the detection agent is the same GDF-8 modulating agent as the unlabeled GDF-8 modulating agent first on the surface of the reaction vessel. In some preferred embodiments, the GDF-8 modulating agent is MYO-029.

In certain embodiments, the methods of the invention enable the detection of antibodies in a biological sample that specifically bind with follistatin, various GDF-8 binding receptors, activin, GDF-8 propeptide, or other GDF-8 modulating agents in biological samples. In other embodiments, the methods enable the detection of antibodies to administered GDF-8 modulating agent in a biological sample from an individual.

The detection agent in steps (c) and (d) is, in some embodiments (e.g., as depicted in FIG. 2), GDF-8 labeled with a detection agent. In some preferred embodiments the detection agent comprises biotin. The methods of this invention also include the detection of antibodies in a biological sample that specifically bind with follistatin, various GDF-8 binding receptors, activin, GDF-8 propeptide, or other GDF-8 modulating agents in biological samples. In other embodiments, this method enables the detection of antibodies to administered therapeutic GDF-8 modulating proteins in a biological sample from an individual.

The invention provides a method to assess an individual's immune response to a first GDF-8 inhibitor, the method comprising: (a) contacting a first GDF-8 inhibitor with a surface of a reaction vessel; (b) adding the biological sample to the reaction vessel; (c) adding a labeled, second GDF-8 inhibitor to the reaction vessel; and (d) detecting a labeled second GDF-8 inhibitor/antibody complex associated with the surface, wherein detection of labeled complex indicates an immune response to the first GDF-8 inhibitor. In some preferred embodiments, the first GDF-8 inhibitor is MYO-029. In some preferred embodiments, the second GDF-8 inhibitor is MYO-029.

Further, a method to assess an individual's immune response to a GDF-8 modulating agent is provided. In one embodiment, the method to assess an individual's immune response to a first GDF-8 inhibitor comprises: (a) contacting a GDF-8 inhibitor with a surface of a reaction vessel; (b) adding the biological sample to the reaction vessel; (c) adding a labeled GDF-8 protein to the reaction vessel; and (d) comparing the amount of labeled GDF-8 protein associated with the surface in the test sample to a control sample, wherein detection of a decreased level of labeled complex indicates an immune response to the GDF-8 inhibitor. In some preferred embodiments, the first GDF-8 inhibitor is MYO-029.

Reporter Gene Assay

In certain other embodiments, the in vitro assay is a reporter gene assay (RGA) (see, e.g., Thies et al., Growth Factors 18:251-259 (2001)). In certain embodiments, an RGA comprises the steps of: (a) providing a host cell comprising a reporter gene construct in a reaction vessel, wherein the construct comprises a GDF-8-responsive control element and a reporter gene; (b) adding an amount of mature GDF-8 protein to the vessel sufficient to activate expression of the reporter gene; (c) adding an amount of a GDF-8 modulating agent to the vessel of step (b) sufficient to modulate the GDF-8 activation of the reporter gene; (d) adding a biological sample to the reaction vessel; and (e) detecting reporter gene expression in the cell in the presence and absence of the biological sample. In some embodiments, the method comprises the further step of adding a substrate that changes color, luminescence, or fluorescence in the presence of the reporter gene.

A host cell may be a eukaryotic cell from a human, mammal or other animal. In a preferred embodiment, the host cell is a cell line, such as a eukaryotic cell line, a mammalian cell line, or a cancer cell line, including a rhabdosarcoma cell line. The report gene contruct may be transiently or stably introduced into the host cell by any means known in the art, including transfection, electroporation, and the like. The reporter gene construct comprises a GDF-8-responsive control element, such as promoter and/or enhancer sequences, and a reporter gene (e.g., capable of expressing a detection agent such as an enzyme) in operative association with the control element (see, for example U.S. Patent Pub. No. 2003/0138422, and references described therein). In preferred embodiments, the enzyme will catalyze the conversion of a substrate to, for example, a calorimetric, fluorescent, or luminescent molecule, and the amount of the reporter gene expression will be assessed by measuring the conversion of substrate to a detectable product, as described above.

For example, to demonstrate the activity of GDF-8, a reporter gene assay (RGA) has been developed using a reporter vector pGL3(CAGA)₁₂ expressing luciferase. The amount of GDF-8 protein added to the assay may be titrated for optimization. An amount of GDF-8 protein is selected that is sufficient to produce 40%, 50%, 60%, 70%, 80%, or 90% of maximal reporter construct activation. GDF-8 protein may be added at 0.05, 0.1, 0.5, 1, 5, 10, 20, 30, 40, 50, 60, 70, 80, 90, 100, 150, 200, 500, or 1,000 ng/mL, for example. Using a constant amount of GDF-8 protein, the GDF-8 modulating agent may be titrated to prepare a titration of modulation of GDF-8 activity. For example, a GDF-8 modulating agent (such as MYO-029) may be tested at concentrations selected from 0.5, 1, 5, 10, 20, 30, 40, 50, 60, 70, 80, 90, 100, 150, 200, 500, or 1,000 ng/mL, for example. In preferred embodiments, a GDF-8 modulating agent titration will span the linear range of inhibition in the assay. To identify an antibody that inhibits a biological activity of a GDF-8 modulating agent, an amount of agent is added to the reaction that is sufficient to inhibit the GDF-8 protein activity by at least 50%, 60%, 70%, 75%, 80%, 85%, 90%, or 95%, for example. An amount of MYO-029 is selected that provides at least about 50% inhibition of the GDF-8 protein-mediated activity, and the biological sample is titrated into the reaction. Optionally, an amount of MYO-029 is selected that inhibits the GDF-8 signal by approximately 80%. The biological sample may then be added in one or more amounts, to identify the ability of an antibody in the sample to overcome the effect of the GDF-8 modulating agent in the reporter gene assay, for example. In preferred embodiments, the agent is pre-incubated with the test sample prior to addition to the assay. To vary the amounts of the biological sample that are added, dilutions of approximately 1:4, 1:5, 1:8, 1:10, 1:15, 1:20, 1:25, 1:30, 1:35, 1:40, 1:45, and/or 1:50 are used, for example. Optionally, the biological sample may be concentrated or fractionated as described above.

Cells are then treated with or without 10 ng/mL GDF-8, for example, and with or without the test biological sample in McCoy's 5A media with glutamine, streptomycin, penicillin, and 1 mg/mL bovine serum albumin for 6 hrs at 37° C. In certain embodiments, GDF-8 modulating agent controls are run in parallel using concentrations from 10 pM to 50 μM, approximately. Exemplary concentrations include 10 pM, 50 pM, 100 pM, 1 nM, 10 nM, 50 nM, 100 nM, 500 nM, 1 μM, 5 μM, 10 μM, and 50 μM of GDF-8 modulating agent; In preferred embodiments, the amount of GDF-8 modulating agent in the test sample is compared to a control titration of known amounts of the agent, and thereby quantitated. Luciferase may be quantified in the treated cells using well known techniques. Alternatively, a reporter construct that is responsive to GDF-8 modulation may be used, optionally comprising a detection marker other than luciferase.

While this disclosure refers to preferred embodiments for detecting antibodies capable of binding to a target GDF-8 modulating agent, it is recognized that antibodies to a variety of target substances may be detected using the methods of the present invention. Similarly, although the disclosure of the present invention is directed to detecting and/or monitoring antiglobulin production in humans in connection with in vivo administration of diagnostic or therapeutic products, it will be recognized that the methodology may be adapted for use in other applications and species as well.

### EXAMPLES

### Example 1

### Assays for the Detection of Antibodies to MYO-029 with Biotinylated MYO-029

Three assays may be performed in the characterization of an immune response to a GDF-8 modulating agent, such as MYO-029: screening, titer, and specificity assays. The protocol for confirming a positive result (e.g., detecting an immune response to MYO-029) initially involves testing all samples in the screening assay format. Screen-negative samples, generating an OD less than the cutpoint, are reported as negative, and are not tested further. Screen-positive samples, i.e., samples generating an OD greater than or equal to the cutpoint, are subsequently tested in titration and specificity assays.

One anti-MYO-029 antibody enzyme-linked immunosorbent assay (ELISA) is a specific example that has been developed for screening, titer, and specificity assays. This embodiment is a bridge assay designed to detect antibodies to the neutralizing GDF-8 modulating antibody MYO-029. The anti-MYO-029 antibody ELISA procedure has been performed for rat, monkey, mouse, rabbit, and human serum samples. In this assay, MYO-029 is adsorbed onto wells of a microtiter plate. Biotinylated MYO-029 is co-incubated with diluted serum samples to allow any GDF-8 modulating agent specific immunoglobulins to bridge between the adsorbed and biotinylated MYO-029 and added to the assay. Bridged biotinylated MYO-029 is detected by an avidin-horseradish peroxidase (HRP) conjugate that produces a colored solution in wells of the plate when 3,3′,5,5′-tetramethylbenzidine (TMB) peroxidase substrate is added. The OD of each well is directly proportional to the amount of biotinylated MYO-029 bound and is determined spectrophotometrically.

Positive and negative controls are run on each plate to monitor assay performance. The ODs of the samples are compared to the cutpoint OD of the plate to determine if antibodies specific for MYO-029 are present. The cutpoint OD is defined as twice the mean of the negative control OD. For human and rabbit serum samples, the cutpoint 1/OD is defined as 1.2 times the mean of the negative control 1/OD (normal human or normal rabbit serum, respectively, diluted 1:8). Samples are initially tested in a screening format at dilutions of 1:8 and 1:16. Any sample generating an OD greater than or equal to the cutpoint is reanalyzed in the titer format (samples diluted serially 1:2 starting from 1:8) to obtain the titer. The antibody titer is defined as the reciprocal dilution of the sample that generates an OD equal to the cutpoint OD. The log of that titer is reported. To identify positive results and confirm specificity of the antibodies for MYO-029, a specificity assay may be performed. Samples that are positive in the titration format are tested in the ELISA on plates that have not been coated with MYO-029 (only coating buffer is added to wells).

Samples that are positive in the titration format and negative in the specificity format are confirmed positive for anti-MYO-029 antibodies, according to the following guidelines: 1) screen-negative samples are reported as negative and not tested further; 2) screen-positive samples are subsequently tested in the titration and specificity ELISAs; 3) the final sample result for a titration-positive sample depends on the specificity result; 4) specificity-negative samples are considered positive (see below) while specificity positive samples may be positive, depending on the magnitude of specificity result.

### Example 2

### Assays to Screen for Antibodies to MYO-029 with Biotinylated MYO-029

Biological samples were initially tested in the screening assay format described in this example. Each well of a 96-well microtiter ELISA plate (high binding, Costar) was coated with 100 μl per well of coating solution (0.5 μg/mL MYO-029 in 100 mM bicarbonate buffer, pH 9.6) the day prior to sample analysis. The plate was covered with sealing film and incubated at 2-8° C. overnight (16-20 hours). The following day, the plate was washed two times with 300 μl/well THST wash buffer (50 mM Tris-HCl, pH 8.0, containing 1.0 mM glycine, 0.5 M NaCl, and 0.05% (v/v) Tween 20®) using an automatic plate washer.

Blocking buffer (Dulbecco's PBS+4% (w/v) nonfat dry milk; 200 μl per well) was then added and the plate was covered with sealing film and incubated at room temperature for 1.5-3.0 hours. The plate was then washed four times with wash buffer (300 μl/well), reversing the plate after the second wash. The washed plate was then either immediately used in the assay or sealed and stored at 2-8° C. for up to four days, where day 1 is defined as the day of blocking.

Human serum samples were thawed at room temperature and mixed thoroughly. Initial dilutions of 1:25 in PBST (Dulbecco's PBS+0.05% (w/v) Tween 20®) and one subsequent 1:3 dilution in PBST+4% (v/v) normal human serum were made. Sample solutions (50 μl/well) were transferred to plate wells in duplicate. Biotinylated MYO-029 (see Example 12) was added to each of the plate wells (50 μl/well). (Positive and negative controls are described further in the following example.) Plates were covered with plate sealing film and incubated on a plate shaker for 2 hours±10 minutes at room temperature. Plates were then washed four times with 300 μl/well wash buffer, reversing the plate after the second wash.

Avidin D-HRP (Vector Laboratories, Burlingame, Calif.) diluted in PBST to a final dilution of 1:50,000 (100 μL/well) was then added to each of the plate wells. Plates were covered and incubated on a plate shaker at room temperature for 1 hour±10 minutes. Plates were then washed six times with wash buffer (300 μL/well), reversing the plate after the third wash.

A solution of the horseradish peroxidase substrate TMB (BioFX Laboratories (Randallstown, Md.)), at room temperature, was then added to each well of the plate (100 μl/well). The plate was incubated in the dark at room temperature for approximately 12±1 minutes before the reaction was quenched by the addition of 0.18 M sulfuric acid (100 μl/well) to each of the plate wells in the same order as that of substrate addition.

The ODs of the samples at a wavelength of 450 nm were compared to the cutpoint OD of the plate to determine if antibodies specific for MYO-029 were present. The cutpoint OD is defined as 1.5 times the mean of the negative control which is normal human serum diluted 1:25. Absorbance at 450 nm was measured with a Molecular Devices Spectra Max 250 plate reader within 30 minutes after quenching the reaction.

For the assay, the mean OD of the negative control is <0.150. The average positive control titer is determined based on the plate cutpoint OD value (the cutpoint is defined as 1.5× mean of the negative control OD) using the equation in Example 7.

Screen-negative samples, generating an OD less than the cutpoint, are reported as negative, with a log titer<1.40, and are not tested further. Screen-positive samples (samples generating an OD greater than or equal to the cutpoint), are subsequently tested in the titration and specificity assays.

### Example 3

### Controls

Negative Control

General Considerations—Pooled normal human serum (e.g., from Bioreclamation Inc. (Hicksville, N.Y.)) was used as a negative control. Negative control solutions were prepared on the day of the experiment by diluting room temperature serum diluted 1:25 with PBST.

Intra-assay Variability—Intra-assay variability (CV) for the OD and cutpoint values of the negative control solution was determined by analysis of the 16 replicate wells on each plate. Three days of testing were evaluated. Data obtained for each plate were analyzed independently in order to generate intra-assay precision results. The mean of the cutpoint values obtained from days 1, 2, and 3 are 0.087, 0.077, and 0.072, respectively. The corresponding CV values are 18.7, 6.4, and 3.7%. The CVs of negative control OD values (16 replicates per plate) ranged between 2.2% and 13.3%.

Inter-assay Variability—Inter-assay variability (CV) for the OD and cutpoint values of the negative control solution was determined. The mean cutpoint OD and corresponding CV value were found to be 0.089 and 25.0%, respectively. The mean OD and corresponding CV value for the negative control were found to be 0.060 and 25.0%, respectively. The CV value for the 16 OD replicates of negative control on each plate ranged between 2.2% and 13.3%.

Positive Control

General Considerations—Affinity-purified goat anti-human IgG antibody (KPL, Gaithesburg, Md.) was used as a positive control for the MYO-029 bridging ELISA assay. Positive control stock solutions were prepared on the day of the experiment by rehydrating 1 mg of goat anti-human IgG in a mixture of 0.5 mL purified water and 0.5 mL glycerol. The stock solution was diluted to 500 ng/mL in PBS+0.1% BSA or 500 ng/mL in PBS+0.1% BSA.

Dilutional Linearity—Dilutional linearity tests were carried out for the positive control solution. Five test solutions were prepared with initial starting dilutions of the positive control of 1:25, 1:75, 1:225, 1:675, and 1:2025. The results demonstrated that the CV between all of the titer values that could be calculated for the five positive control titrations tested was 1.6%. There was no trend toward non-linearity detected in the test.

Intra-assay Variability—Intra-assay variability was determined for the OD and titer values of the positive control solutions. The positive control was tested multiple times on the same plate (intra-assay). Each plate contained 5 individual positive control titrations. The test was performed using a total of 4 plates on day 1, 2 plates on day 2, and 4 plates on day 3. Intra-assay variability was evaluated on three separate days. Data obtained for each plate were analyzed independently in order to generate intra-assay precision results.

The between plate variability of the maximum OD and titer values for the positive control was determined employing data obtained from all plates tested per day. The intra-assay (intra-plate) CVs obtained for the log titers were within 2.2%. The CV values obtained using the individual positive control log titers over all plates tested for days 1, 2, and 3 are 12.4, 1.7, and 1.5%, respectively. The CV values obtained for the maximum OD generated by the positive control over all plates tested for days 1, 2, and 3 is 18.9, 2.8, and 3.2%, respectively.

The mean SD and CV of OD values at each dilution of the positive control over 4 plates were analyzed for day 3 of the intra-assay evaluation. The CV values ranged between 2.8% and 4.7%. A titration profile of the positive control titration was performed. There was no evidence of a prozone effect.

Inter-assay Variability—To determine the random inter-assay variability of the positive control OD values and titers, the control solutions were analyzed on 20 plates over 6 days. For each plate, one set of duplicates of the positive controls (same position on each plate) was used during final data analysis. For the positive control solution, mean log titer and log titer CV values were 3.37 and 6.6%, respectively. The mean maximum OD and maximum OD CV values were 2.137 and 11.9%, respectively.

Formulation—The positive control working stock solution (500 ng/mL) was initially prepared in deionized water with 0.1% BSA for evaluation. All of the validation runs were carried out using the working stock prepared in deionized water+0.1% BSA. The positive control working stock solution was then subsequently prepared in PBS with 0.1% BSA since PBS was the desired diluent. A comparison of the log titer and the maximum OD of both solutions was performed by analyzing the positive control prepared from the two different stock solutions on the same plate run. No significant difference in the log titers for the positive control was observed (data not shown). The difference in maximum OD and numerical titer can be considered within the assay variability. The working stock solution will be prepared in the PBS+0.1% BSA and stored at −70° C. for up to 1 year.

### Example 4

### Reactivity in the Unexposed Population

Using the procedure of Example 1, twenty-five individual normal serum samples were tested three times over 3 days (n=75 results) in order to analyze statistical distribution of ODs and determine a statistically based assay cutpoint value.

The average value of sample ODs for the 25 samples analyzed over 3 days was 0.058, which is identical to that for the average OD value for negative controls analyzed on the same plates. This indicates that the negative control performance is representative of normal individual sample performance and can be used to normalize the cutpoint ODs between plates. Therefore the cutpoint for an individual plate can be calculated by multiplying the plate negative control mean OD by a multiplication factor n that was derived based on the estimated 95^{th} percentile for the 25 normal samples.

The nonparametric estimate of the 95% percentile was made by analyzing the generated collection of the OD values and determining an OD high enough to include 95% of the values (0.081). The ratio of the 95% percentile to the mean of the samples and the negative control OD was 1.39. Since this assessment was performed using a limited sample set tested over a short time period (3 consecutive days), the variability in performance of clinical study samples, collected from multiple sites and analyzed over a longer period of time, is expected to be wider. Taking the expected higher variability of study samples into consideration and for convenience, the multiplication factor n to be used for calculation of the plate cutpoint value was rounded up from 1.39 to 1.5.

### Example 5

### Assay Sensitivity

Affinity purified, goat anti-human IgG was used to prepare the positive control solution. Hence, assay sensitivity was calculated based on the initial positive control reagent concentration and its numerical titer. In the assay, the starting concentration of the positive control reagent was 500 ng/mL. The average titer value obtained during the evaluation done above for the positive control solution prepared in PBS+0.1% BSA was 4680. The assay sensitivity was determined to be 107 pg/mL (500 ng/mL/4680) using the following equation:

$$Sensitivity = \frac{Starting concentration of the positive control solution}{Positive control numerical titer}$$

### Example 6

### Test of Drug Interference

The positive control solution (1:25) was spiked with various amounts of MYO-029 to give a final concentration of the drug at 0.01, 0.1, 1.0, and 10 μg/mL prior to the subsequent 3-fold dilutions in PBST (1:75, 1:225, 1:675, 1:2025, 1:6075, 1:18225, and 1:54675). The titers and maximum ODs are shown in Table 2.

TABLE 2

| | | Log Title | | |
|---|---|---|---|---|
| | Log | % | Max. | Max. OD |
| Assay Condition | Titer | Difference | OD | % Difference |
| No Spike | 3.12 | | 2.460 | |
| Spiked at 0.01 μg/mL MYO-029 | 2.83 | −9.3 | 1.194 | −51.5 |
| Spiked at 0.1 μg/mL MYO-029 | 2.16 | −30.8 | 0.189 | −92.3 |
| Spiked at 1.0 μg/mL MYO-029 | <1.40 | NA | 0.079 | −96.8 |
| Spiked at 10 μg/mL MYO-029 | <1.40 | NA | 0.061 | −97.5 |

The results showed that the performance of the positive control was significantly affected at 0.1 μg/mL and higher concentrations of MYO-029 in the sample. A drop in the maximum OD was observed for the 0.01 μg/mL concentration of MYO-029. Due to the heterogeneity of the antibody response, interference of MYO-029 found in samples can be different from the interference detected for the positive control.

### Example 7

### Titration Assays

Samples are initially tested in a screening format at dilutions of 1:25 and 1:75. Any sample generating an OD greater than or equal to the cutpoint is reanalyzed in the titer and specificity assays (along with the corresponding pre-dose sample, if the sample generating an OD greater than or equal to the cutpoint is a post-dose sample). Titer and specificity assays for the sample may be performed simultaneously or sequentially. To obtain the antibody titer, samples are first diluted 1:25 and then diluted serially 1:3 with PBST+4% human serum from 1:25 to obtain dilutions through 1:54,675. Each sample is assayed in duplicate. The assay is performed essentially as described in Example 2.

The titer of a given sample is defined as the reciprocal dilution of the sample that would generate an OD value equal to the cutpoint. Numerical titer values are calculated by interpolation using Equation 1 below, where ODcp is the cutpoint OD, OD1 is the sample OD value above the cutpoint OD in the dilution series, OD2 is the sample OD value below the cutpoint OD in the dilution series, DilnOD1 is the sample reciprocal dilution at OD1, and DilnOD2 is the sample reciprocal dilution at OD2.

$$[ Titer ] = [ DilnOD 1 - ( ( \frac{OD 1 - ODcp}{OD 1 - OD 2} ) * ( DilnOD 1 - DilnOD 2 ) ) ]$$

The logarithmic value of the numerical titer is reported as the final result.

$$log [ Titer ] = log [ DilnOD 1 - \{ ( \frac{OD 1 - ODcp}{OD 1 - OD 2} ) * ( DilnOD 1 - DilnOD 2 \} ]$$

For example, in the analysis of a rat serum sample shown in Table 3 below, ODcp=0.252, Diln OD1=675, and Diln OD2=2025, and the log titer of the unknown sample is 3.29. Raw data were analyzed using Watson LIMS system.

TABLE 3

| Positive control | | Mean | Negative control | | Mean | Unknown | | Unk |
|---|---|---|---|---|---|---|---|---|
| (PC) | | PC | (NC) | | NC | sample | | mean |
| 2.974 | 2.882 | 2.928 | 0.121 | 0.122 | 0.126 | 1.684 | 1.678 | 1.681 |
| 1.402 | 1.400 | 1.401 | 0.129 | 0.129 | | 1.665 | 1.678 | 1.672 |
| 0.610 | 0.676 | 0.643 | 0.129 | 0.132 | | 1.150 | 1.149 | 1.150 |
| 0.330 | 0.334 | 0.332 | 0.120 | 0.130 | | 0.440 | 0.506 | 0.473 |
| 0.209 | 0.197 | 0.203 | 0.116 | 0.128 | | 0.258 | 0.216 | 0.237 |
| 0.164 | 0.197 | 0.181 | 0.125 | 0.119 | | 0.187 | 0.148 | 0.168 |
| 0.151 | 0.146 | 0.148 | 0.126 | 0.120 | | 0.144 | 0.121 | 0.132 |
| 0.140 | 0.153 | 0.147 | 0.131 | 0.141 | | 0.139 | 0.133 | 0.136 |

### Example 8

### Specificity Assay

Samples were initially tested in a screening format at dilutions of 1:25 and 1:75. Positive samples were tested. To confirm specificity of the antibodies for MYO-029, samples that are positive in the screen or titration format are tested on plates that have not been coated with MYO-029 (only coating buffer is added to wells).

Samples are diluted serially 1:3 with PBST+4% human serum from 1:25 to obtain dilutions through 1:54,675. Each sample is assayed in duplicate. The assay is performed as described in Example 1. Each well of a 96-well microtiter ELISA plate (Costar) was coated with 100 μl per well of coating buffer (100 mM bicarbonate buffer, pH 9.6) the day prior to sample analysis. The plate was covered with sealing film and incubated at 2-8° C. overnight (16-20 hours). The following day, the plate was washed two times with 300 μl/well wash buffer using an automatic plate washer.

Blocking buffer (Dulbecco's PBS+4% (w/v) nonfat dry milk; 200 μl per well) was then added and the plate covered with sealing film and incubated at room temperature for 1.5-3.0 hours. The plate was then washed four times with wash buffer (300 μl/well), reversing the plate after the second wash. The washed plate was then either immediately used in the assay or sealed and stored at 2-8° C. for up to four days, where day 1 is defined as the day of blocking.

Samples were thawed at room temperature and mixed thoroughly. Initial dilutions of 1:25 in PBST (Dulbecco's PBS+0.05% (w/v) Tween 20) and one subsequent 1:3 dilution in PBST+4% (v/v) normal human serum were made. Biotinylated MYO-029 was added to each of the plate wells (50 μl/well) in duplicate. (Positive and negative controls are described further in the following example.) Sample solutions (50 μl/well) were transferred to plate wells in duplicate. Plates were covered with plate sealing film and incubated on the plate shaker for 2 hours±10 minutes at room temperature. Plates were then washed four times with 300 μl/well wash buffer, reversing the plate after the second wash.

Avidin D-HRP (Vector Laboratories, Burlingame, Calif.) solution (100 μL/well) was then added to each of the plate wells. Plates were covered and incubated on a plate shaker at room temperature for 1 hour±10 minutes. Plates were then washed six times with wash buffer (300 μL/well), reversing the plate after the third wash.

A solution of horseradish peroxidase substrate 3,3′,5,5′-tetramethylbenzidine (TMB substrate; BioFX Laboratories (Randallstown, Md.)), at room temperature, was then added to each well of the plate (100 μl/well). The plate was incubated in the dark at room temperature for approximately 12±1 minutes before the reaction was quenched by the addition of 0.18 M sulfuric acid (100 μl/well) to each of the plate wells in the same order as that of substrate addition.

The ODs of the samples are compared to the cutpoint OD of the plate to determine if the signal from the titration assay detects antibodies specific for MYO-029. The cutpoint OD is defined as 1.5 times the mean of the negative control. Absorbance at 450 nm was measured within 30 minutes after quenching the reaction.

The final sample result for a titration-positive sample depends on the specificity result (e.g., specificity-negative samples are considered positive). Samples that are positive in the titration format and negative in the specificity format are confirmed positive for anti-MYO-029 antibodies. Specificity positive samples may or may not be considered positive, depending upon the magnitude of specificity result.

### Example 9

### Sample Assessment

Titer and specificity assay results are assessed based upon the following table. In cases where a repeat of the titer assay produces a result that is incongruent with the original result, the default is the original result.

TABLE 4
Sample Assessment

| | | Re-Assay | | |
|---|---|---|---|---|
| Screen | Titer | Titer | | |
| Result | Result | Result | Corresponding Specificity | Reported Result |
| (Log Titer) | (Log Titer) | (Log Titer) | Plate Result (Log Titer) | (Log Titer) |
| <1.40 | NA | NA | NA | Negative (<1.40) |
| ≧1.40 | <1.40 | NA | NA | Negative (<1.40) |
| and | ≧1.40 | NA | <1.40 | Positive (TR) |
| ≦1.88 | and <0.48 | | ≧1.40 and TR ≧0.48 above SR | Positive (TR) |
| | above ScR | | ≧1.40 and TR <0.48 above SR | Negative (<1.40) |
| | ≧1.40 | <1.40 | <1.40 | Positive (TR) |
| | and ≧0.48 | | ≧1.40 and TR ≧0.48 above SR | Positive (TR) |
| | above | | ≧1.40 and TR <0.48 above SR | Negative (<1.40) |
| | ScR. | | | |
| | Repeat | ≧1.40 | <1.40 | Positive (RTR) |
| | titer assay. | | ≧1.40 and RTR ≧0.48 above SR | Positive (RTR) |
| | | | ≧1.40 and RTR <0.48 above SR | Negative (<1.40) |
| >1.88 | <1.40 | <1.40 | NA | Negative (<1.40) |
| | Repeat | ≧1.40 | <1.40 | Positive (RTR) |
| | titer assay. | | ≧1.40 and RTR ≧0.48 above SR | Positive (RTR) |
| | | | ≧1.40 and RTR <0.48 above SR | Negative (<1.40) |
| | ≧1.40 | NA | <1.40 | Positive (TR) |
| | | | ≧1.40 and TR ≧0.48 above SR | Positive (TR) |
| | | | ≧1.40 and TR <0.48 above SR | Negative (<1.40) |

NA = Not applicable.
ScR = Screening result.
TR = Titer result.
RTR = Re-assay titer result.
SR = specificity assay result.

The determination of whether an antibody to MYO-029 has occurred in the subject is made based on the comparison of the pre- and post-dose sample results. If the pre-dose samples are negative and the corresponding post-dose samples are positive, the subject is considered to be positive for an immune response. If both the pre-dose and post-dose samples test positive, the subject is called positive for an immune response when the post-dose sample titer value is at least one dilution factor (3-fold) higher than the titer value determined for the corresponding pre-dose sample.

### Example 10

### Detection of Antibodies to MYO-029 with Biotinylated GDF-8

To detect antibodies that inhibit the binding of biotinylated GDF-8 to MYO-029, each well of a 96-well microtiter ELISA plate (Costar) was coated with 100 μl per well of coating solution (6 μg/mL MYO-029 in 100 mM bicarbonate buffer, pH 9.6) the day prior to sample analysis. Alternatively, 0.5 μg/mL MYO-029 was used. The plate was covered with sealing film and incubated at 2-8° C. overnight (16-22 hours). The following day, the plate was washed two times with 300 μl/well wash buffer using an automatic plate washer.

Blocking buffer (Dulbecco's PBS+1% (w/v) bovine serum albumin; 250 μl per well) was then added, and the plate was covered with sealing film and incubated at room temperature for 1.5-3.0 hours. The plate was then washed four times with 300 μl/well THST wash buffer (50 mM Tris-HCl, pH 8.0, containing 1.0 mM glycine, 0.5 M NaCl, and 0.05% (v/v) Tween 20®), reversing the plate after the second wash. The washed plate was then either immediately used in the assay or sealed and stored at 2-8° C. for up to four days, where day 1 is defined as the day of blocking.

Samples were thawed at room temperature and mixed thoroughly. Initial dilutions of 1:8 in THST and one subsequent 1:2 dilution in THST were made. Sample solutions (100 μl/well) were transferred to plate wells in duplicate. Alternatively, initial sample dilutions of 1:25 in PBST (Dulbecco's PBS 0.05% w/v Tween 20) followed by a 1:3 dilution were made, transferring 50 μl/well.

A positive control stock solution (rabbit anti-MYO-029 antiserum, spiked into normal human serum at 1:6.25 dilution) was thawed and diluted 1:8 in THST. Two-fold serial dilutions were subsequently made in THST containing 12.5% pooled normal human serum, yielding the following set of positive control dilutions: 1:8, 1:16, 1:32, 1:64, 1:128, 1:256, 1:512, 1:1024. Positive control solutions (100 μl/well) were transferred to plate wells in duplicate. Plates were covered with sealing film and incubated on the plate shaker for 2 hours±10 minutes at room temperature. Plates were then washed four times with 300 μl/well THST wash buffer, reversing the plate after the second wash.

Biotinylated GDF-8 (see Example 12) was then added to each of the plate wells (100 μl/well of a 35 ng/mL solution). Plates were covered with plate sealing film and incubated on the plate shaker for 1.5 hours±10 minutes at room temperature. Plates were then washed four times with 300 μl/well wash buffer, reversing the plate after the second wash.

Avidin-HRP (Pierce, Rockford, Ill.) solution (100 μL/well) was then added to each of the plate wells. Avidin D-HRP (Vector Laboratories, Burlingame, Calif.) was alternatively used. Plates were covered and incubated on a plate shaker at room temperature for 40 minutes or an hour. Plates were then washed four times with wash buffer (300 μL/well), reversing the plate after the second wash.

A solution of peroxidase substrate 3,3′,5,5′-tetramethylbenzidine (TMB substrate; BioFX Laboratories, Randallstown, Md.), at room temperature, was then added to each well of the plate (100 μl/well). The plate was incubated in the dark at room temperature for approximately 10 minutes before the reaction was quenched by the addition of 0.18 M sulfuric acid (100 μl/well) to each of the plate wells in the same order as that of substrate addition. The optical density was read at 450 nm on a spectrophotometer (Molecular Devices, Sunnyvale, Calif.) within 30 minutes of quenching, and ODs were transformed to 1/OD for analysis.

### Example 11

### Determination of Titer and Data Analysis

For samples testing positive in the above screening assay (Example 10), the titer was determined by diluting test samples 1:2 in THST followed by seven subsequent 1:8 serial dilutions in THST containing 12.5% pooled normal human serum, yielding final dilutions of 1:8, 1:16, 1:32, 1:64, 1:128, 1:256, 1:512, and 1:1024. The titer of a given sample is defined as the reciprocal dilution of the sample that would generate a 1/OD value equal to the cutpoint. The cutpoint of the assay is defined as 1.2 times the mean negative control 1/OD value; and is based on the 95^{th} percentile of 1/OD values observed in a panel of serum samples from normal human individuals. Numerical titer values are calculated by interpolation using the equation below, where ODcp is the cutpoint OD, OD1 is the sample OD value above the cutpoint OD in the dilution series, OD2 is the sample OD value below the cutpoint OD in the dilution series, DilnOD1 is the sample reciprocal dilution at OD1, and DilnOD2 is the sample reciprocal dilution at OD2.

$$[ Titer ] = [ Diln 1 / OD 1 - ( ( \frac{1 / OD 1 - 1 / ODcp}{1 / OD 1 - 1 / OD 2} ) * ( Diln 1 / OD 1 - Diln 1 / OD 2 ) ) ]$$

Titer and specificity assay results were assessed using the following table. In cases where a repeat of the titer assay produces a result that is incongruent with the original result, the default is the original result.

TABLE 5

| Screen | | Re-Assay | | |
|---|---|---|---|---|
| Result | Titer Result | Titer Result | Corresponding Specificity | Reported Result |
| (Log Titer) | (Log Titer) | (Log Titer) | Plate Result (Log Titer) | (Log Titer) |
| <0.903 | NA | NA | NA | Negative (<0.903) |
| ≧0.903 | <0.903 | NA | NA | Negative (<0.903) |
| and | ≧0.903 | NA | <0.903 | Positive (TR) |
| ≦1.50 | and <0.60 | | ≧0.903 and TR ≧0.60 above SR | Positive (TR) |
| | above ScR | | ≧0.903 and TR <0.60 above SR | Negative (<0.903) |
| | ≧0.903 | <0.903 | <0.903 | Positive (TR) |
| | and ≧0.60 | | ≧0.903 and TR ≧0.60 above SR | Positive (TR) |
| | above ScR. | | ≧0.903 and TR <0.60 above SR | Negative (<0.903) |
| | Repeat titer | ≧0.903 | <0.903 | Positive (RTR) |
| | assay. | | ≧1.05 and RTR ≧0.60 above SR | Positive (RTR) |
| | | | ≧1.50 and RTR <0.60 above SR | Negative (<0.903) |
| >1.50 | <0.903 | <0.903 | NA | Negative (<0.903) |
| | Repeat titer | ≧0.903 | <1.40 | Positive (RTR) |
| | assay. | | ≧0.903 and RTR ≧0.60 above SR | Positive (RTR) |
| | | | ≧0.903 and RTR <0.60 above SR | Negative (<0.903) |
| | ≧0.903 | NA | <0.903 | Positive (TR) |
| | | | ≧0.903 and TR ≧0.60 above SR | Positive (TR) |
| | | | ≧0.903 and TR <0.60 above SR | Negative (<0.903) |

NA = Not applicable.
ScR = Screening result.
TR = Titer result.
RTR = Re-assay titer result.
SR = specificity assay result.

### Example 12

### Biotinylation

GDF-8 was biotinylated as follows. Full length GDF-8 was expressed in a fed-batch CHO cell culture bioreactor process, providing the latent complex form of GDF-8. The cell culture harvest was clarified using normal flow microporous filtration and then concentrated and diafiltered using tangential flow ultrafiltration. This retentate pool was then loaded onto Ni²⁺-NTA immobilized metal affinity chromatography (IMAC) where the GDF-8 complex is captured. Elution occurred with a 50 mM Na₂HPO₄, 300 mM NaCl, 20-500 mM imidazole linear gradient over 5 column volumes. The resulting peak then underwent buffer-exchange via dialysis to allow IMAC-derived imidazole removal and to put an appropriate buffer in place for the biotinylation reaction.

The latent complex preparation was then biotinylated. A target sulfo-NHS-LC-biotin to GDF-8 complex molar ratio of 14:1 was used in the reaction. Reagent to substrate ratios of 10:1, 15:1, and 20:1 have also been tested, for example. Solid biotin reagent (EZ-link Sulfo-NHS-Biotin, Pierce Biotechnology) was dissolved in dimethyl sulfoxide (DMSO) at 200 g/L before it was added to the GDF-8 complex sample. The reaction was performed with a GDF-8 complex concentration of less than 1.5 g/L in 100 mM Na₂HPO₄, 150 mM NaCl, pH 7.2, at 4° C., for 120 minutes. The reaction mixture was mixed gently at the start of the reaction and shielded from light during the course of the reaction. The reaction was stopped by adding 0.5% (v/v) ethanol amine or 5.0% (v/v) 1 M Tris.

This biotinylated GDF-8 complex was then buffer-exchanged via dialysis into a low pH, high chaotrope concentration buffer (6000 mM urea, 300 mM NaCl, 50 mM H₃PO₄, pH=2.5). Dissociation of the complex occurs with protonation at low pH. In this buffer, the complex dissociates and solubilizes into propeptides and mature dimers. Also, free biotin is removed during the dialysis. This retentate pool was then loaded onto high performance size exclusion chromatography where the mature dimer form of GDF-8 is separated from propeptides and residual monomer.

This fraction comprising the biotinylated, mature dimer form of GDF-8 was then further processed on butyl high performance reversed phase chromatography using a 0-90% (v/v) CH₃CN, 0.1% (v/v) CF₃CO₂H, pH=2.0 linear gradient over 5 column volumes. The peak from this step was buffer-exchanged via dialysis into a low pH formulation buffer (0.1% (v/v) CF₃CO₂H, pH=2.0).

The biotinylated mature GDF-8 dimer was assessed for retention of function, for example its activity in binding and reporter gene assays. The biotinylated mature GDF-8 protein was measured by reversed-phase high performance liquid chromatography/electrospray-ionization quadrupole time-of-flight mass spectrometry (RP-HPLC/ESI-QTOF-MS), and the preparation contains a mix of molar ratios of approximately 0-3, with the majority of the molecules being at 1:1. Higher target molar ratios have yielded measurements as high as 9:1, by adjustment of conditions well known in the art.

MYO-029 is biotinylated using a similar assay, and may be used in the methods described herein. Essentially, isolated MYO-029 is diluted, buffer-exchanged, and then biotinylated. The reaction and storage conditions are the same as for GDF-8, except for a few parameters. The MYO-029 concentration value ranges from 10-24 g/L. A target sulfo-NHS-LC-biotin to MYO-029 molar ratio in the biotinylation reaction is 40:1, which yields a measured molar ratio of 8-11. This is measured by an avidin:HABA A₆₀₀ nm spectrophotometry assay (Immunopure Avidin and HABA, Pierce). Using dialysis, this reagent is then buffer-exchanged into a low salt, neutral pH formulation buffer (137 mM NaCl, 1 mM KCl, 8 mM Na₂HPO₄, 3 mM KH₂PO₄, pH=7.2).

### Example 13

### Reporter Gene Assay

An antibody that specifically binds to a GDF-8 modulating agent is detected in cell based reporter gene assay (RGA) for biological activity of GDF-8. Antibodies that inhibit the activity of a GDF-8 modulating agent, such as antibodies that neutralize MYO-029 activity, are detected by the following assay.

The human rhabdomyosarcoma cell line A204 pCAGA was used, in which A204 (ATCC HTB-82) was stably transfected with a reporter gene construct, pGL3(CAGA)₁₂ (described in U.S. Patent Publ. Nos. 2003/0138422 A1 and 2004/0142382 A1) using well known techniques. Alternatively, A204 cells are transiently transfected with pGL3(CAGA)₁₂ using FuGENE.TM.6 transfection reagent (Boehringer Manheim, Germany). Following transfection, cells were cultured on 96 well plates in McCoy's 5A medium supplemented with 2 mM glutamine, 100 U/mL streptomycin, 100 μg/mL penicillin and 10% fetal calf serum for 16 hours. Cells were treated with or without a constant amount (75 ng/mL of mature GDF-8 protein, a constant amount of (400 ng/mL) MYO-029 and a dilution series of positive control in McCoy's 5A media with glutamine, streptomycin, penicillin, and 10% fetal calf serum for 6 hours at 37° C. for controls. Optionally, an amount of GDF-8 is selected that provides approximately 80% of the maximal luciferase signal. MYO-029 is preincubated with the GDF-8 at contractions from 6.25 ng/mL to 400 ng/mL (5.9 nM to 375 nM) for 1 hour at room temperature, and then the proteins are added in the RGA. Positive control antibodies to the GDF-8 modulating agent were incubated with MYO-029 and assayed in the RGA. Optionally, an amount of MYO-029 is selected that inhibits the GDF-8 signal by approximately 80%. Luciferase was quantified in the treated cells using the Luciferase Assay System (Promega). In this assay, 75 ng/mL GDF-8 provides 80% activation while 400 ng/mL of MYO-029 provides 80% inhibition of the reporter gene construct.

In parallel reactions, cells are treated with and without 75 ng/mL of mature GDF-8 protein, with and without MYO-029 (or other GDF-8 modulating agent) and with and without test biological samples. Human serum is obtained from individuals undergoing MYO-029 treatment, and diluted 1:5, 1:10, 1:15, 1:20, and 1:40 in buffer. For dilutions lower than 1:10, the test sample serum is further diluted in buffer containing 10% human serum (Bioreclamation, Inc.).

A functional cell based assay was performed based on a published GDF-8 responsive reporter gene assay (U.S. Patent Pub. No. 2003/0138422 A1) as follows: In this assay, 75 ng/mL GDF-8 was preincubated with 400 ng/mL MYO-029 (375 nM) and added to A204 cells transferred with PGL3 (pCAGA)₁₂ cells in the presence and absence of a human serum sample. The human serum sample was diluted 1 to 20, and compared to a positive control comprising rabbit anti-MYO-029 polyclonal serum diluted 1:100, 1:200, 1:400, 1:800, 1:1600, 1:3200, and 1:6400, for example. The assay can be run either mixing together human serum sample or positive control rabbit antibody with MYO-029 then GDF-8 and adding directly to the cells in the RGA or preincubating human serum samples or positive control rabbit antibody with MYO-029 for 1 hour adding to the cells in the RGA then adding GDF-8 to the sample wells.

A positive control of neutralizing antibodies to MYO-029 was developed as follows. Rabibits were immunized with either intact MYO-029 or MYO-029 protein fragments comprising the MYO-029 binding site. The digestion was performed to remove the Fc portion of the MYO-029 antibody in order to avoid generation of a strong immune response in the rabbit to the constant region of this human antibody. Two rabbits were immunized with either the intact or the digested MYO-029. Bleeds were tested for neutralizing activity using ligand binding assays. All four animals developed good antibody titer results and a control rabbit serum was produced by pooling bleeds from all four animals.

All publications, patents, and biological sequences cited in this disclosure are incorporated by reference in their entirety. To the extent the material incorporated by reference contradicts or is inconsistent with the present specification, the present specification will supersede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

The embodiments within the specification provide an illustration of embodiments of the invention and should not be construed to limit the scope of the invention. The skilled artisan readily recognizes that many other embodiments are encompassed by the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method to detect an antibody that specifically binds to a GDF-8 modulating agent in a biological sample, comprising:
(a) adding the GDF-8 modulating agent to an in vitro assay for a GDF-8 activity in a reaction vessel;
(b) adding the biological sample to the in vitro assay for a GDF-8 activity in the reaction vessel;
(c) detecting modulation of the GDF-8 activity by the biological sample; and
(d) comparing the modulation of the GDF-8 activity in the presence of the biological sample to the modulation of the GDF-8 activity in the presence of the GDF-8 modulating agent alone.

2. The method of claim 1, wherein the in vitro assay is an immunoassay comprising:
(a) contacting the GDF-8 modulating agent with a surface of the reaction vessel;
(b) subsequently adding the biological sample to the reaction vessel;
(c) adding a detection agent to the reaction vessel; and
(d) detecting a GDF-8 modulating agent/antibody complex associated with the surface.

3. The method of claim 2, wherein the detection agent is the GDF-8 modulating agent with a detectable label.

4. The method of claim 2, wherein the detection agent is a labeled GDF-8 protein.

5. The method of claim 4, wherein the label is biotin.

6. The method of claim 5, wherein the ratio of moles of biotin incorportated to moles of agent is less than 5:1.

7. The method of claim 5, wherein the ratio of biotin to agent is between about 0.5:1 to 4:1.

8. A method to detect an antibody that specifically binds to a GDF-8 modulating agent in a biological sample, comprising:
(a) contacting the GDF-8 modulating agent with a surface of a reaction vessel;
(b) adding the biological sample to the reaction vessel;
(c) adding a detection agent to the reaction vessel; and
(d) detecting a GDF-8 modulating agent/antibody complex associated with the surface of the reaction vessel.

9. The method of claim 8, wherein the detection agent is the GDF-8 modulating agent of step (a) with a detectable label.

10. The method of claim 8, wherein the detection agent is a labeled GDF-8 protein.

11. The method of claim 10, wherein the GDF-8 modulating agent/antibody complex is detected by comparing GDF-8 modulating agent/labeled GDF-8 protein complex levels in the test sample to levels in a control sample.

12. The method of claim 8, wherein the GDF-8 modulating agent is a GDF-8 inhibitor.

13. The method of claim 12, wherein the GDF-8 inhibitor is an antibody.

14. The method of claim 13, wherein the antibody specifically binds to GDF-8.

15. The method of claim 14, wherein the antibody is MYO-029.

16. The method of claim 8, wherein the GDF-8 modulating agent is chosen from:
(a) an antibody that specifically binds to GDF-8;
(b) an antibody that specifically binds to a GDF-8 binding partner;
(c) a soluble GDF-8 receptor;
(d) an ActRIIB protein;
(e) a follistatin-domain containing protein;
(f) a follistatin protein;
(g) a GASP-1 protein;
(h) a GDF-8 protein;
(i) a GDF-8 propeptide;
(j) a non-proteinacious inhibitor;
(k) a nucleic acid; and
(l) a small molecule.

17. The method of claim 8, wherein the biological sample is from a mammal, bird, reptile, or fish.

18. The method of claim 17, wherein the biological sample is from a mammal.

19. The method of claim 18, wherein the mammal is a human.

20. The method of claim 8, wherein the biological sample is chosen from serum, blood, plasma, biopsy sample, tissue sample, cell suspension, saliva, oral fluid, cerebrospinal fluid, amniotic fluid, milk, colostrum, mammary gland secretion, lymph, urine, sweat, lacrimal fluid, gastric fluid, synovial fluid, and mucus.

21. The method of claim 20, wherein the biological sample is chosen from serum, blood, and plasma.

22. The method of claim 10, wherein the label is chosen from an enzyme, an epitope tag, a radiolabel, biotin, a dye, a fluorescent tag label, and a luminescent label.

23. The method of claim 22, wherein the label is biotin.

24. The method of claim 23, wherein the ratio of moles of biotin incorportated to moles of detection agent is less than 5:1.

25. The method of claim 23, wherein the ratio of biotin to agent is between about 0.5:1 to 4:1.

26. The method of claim 23, further comprising adding an avidin-enzyme conjugate.

27. The method of claim 26, further comprising adding a substrate that changes color, luminescence, or fluorescence in the presence of the enzyme.

28. A method to detect an antibody that specifically binds to a GDF-8 inhibitor in a biological sample, comprising:
(a) contacting a first GDF-8 inhibitor with a surface of a reaction vessel;
(b) adding the biological sample to the reaction vessel;
(c) adding a labeled second GDF-8 inhibitor to the reaction vessel; and
(d) detecting the labeled second GDF-8 inhibitor associated with the surface.

29. The method of claim 28, wherein the biological sample is from a mammal, bird, reptile, or fish.

30. The method of claim 29, wherein the biological sample is from a mammal.

31. The method of claim 30, wherein the mammal is a human.

32. The method of claim 28, wherein the biological sample is chosen from serum, blood, plasma, biopsy sample, tissue sample, cell suspension, saliva, oral fluid, cerebrospinal fluid, amniotic fluid, milk, colostrum, mammary gland secretion, lymph, urine, sweat, lacrimal fluid, gastric fluid, synovial fluid, and mucus.

33. The method of claim 32, wherein the biological sample is chosen from serum, blood, and plasma.

34. The method of claim 28, wherein the first GDF-8 inhibitor and the second GDF-8 inhibitor are the same.

35. The method of claim 28, wherein the first GDF-8 inhibitor is an antibody that specifically binds to GDF-8.

36. The method of claim 28, wherein the second GDF-8 inhibitor is an antibody that specifically binds to GDF-8.

37. The method of claim 28, wherein the label is chosen from an enzyme, an epitope tag, a radiolabel, biotin, a dye, a fluorescent tag label, and a luminescent label.

38. The method of claim 28, wherein the label is biotin.

39. The method of claim 38, further comprising adding an avidin-enzyme conjugate.

40. The method of claim 39, further comprising adding a substrate that changes color, luminescence, or fluorescence in the presence of the enzyme.

41. A method to detect an antibody that specifically binds to MYO-029 in a biological sample, comprising:
(a) contacting isolated MYO-029 with a surface of a reaction vessel;
(b) adding the biological sample to the reaction vessel;
(c) adding labeled MYO-029 to the reaction vessel; and
(d) detecting labeled MYO-029 associated with the surface.

42. A method to detect an antibody that specifically binds to MYO-029 in a biological sample, comprising:
(a) providing a host cell comprising a reporter gene construct in a reaction vessel, wherein the construct comprises a GDF-8-responsive control element and a reporter gene;
(b) adding an amount of mature GDF-8 protein to the vessel sufficient to activate expression of the reporter gene;
(c) adding an amount of MYO-029 to the vessel of step (b) sufficient to modulate the GDF-8 activation of the reporter gene;
(d) adding a biological sample to the reaction vessel of step (c); and
(e) detecting reporter gene expression in the presence and absence of the biological sample.

43. The method of claim 41, wherein the biological sample is from a mammal, bird, reptile, or fish.

44. The method of claim 43, wherein the biological sample is from a mammal.

45. The method of claim 44, wherein the mammal is a human.

46. The method of claim 41, wherein the biological sample is chosen from serum, blood, plasma, biopsy sample, tissue sample, cell suspension, saliva, oral fluid, cerebrospinal fluid, amniotic fluid, milk, colostrum, mammary gland secretion, lymph, urine, sweat, lacrimal fluid, gastric fluid, synovial fluid, and mucus.

47. The method of claim 46, wherein the biological sample is chosen from serum, blood, and plasma.

48. The method of claim 41, wherein the label is chosen from an enzyme, an epitope tag, a radiolabel, biotin, a dye, a fluorescent tag label, and a luminescent label.

49. The method of claim 48, wherein the label is biotin.

50. The method of claim 49, wherein the median ratio of moles of biotin incorporated to moles of agent is at least 5:1.

51. The method of claim 49, wherein the median ratio of biotin to agent is at least 10:1.

52. The method of claim 49, further comprising adding an avidin-enzyme conjugate.

53. The method of claim 52, further comprising adding a substrate that changes color, luminescence, or fluorescence in the presence of the enzyme.

54. A method to detect an antibody that specifically binds to MYO-029 in a biological sample, comprising:
(a) contacting isolated MYO-029 with a surface of a reaction vessel;
(b) adding the biological sample to the reaction vessel;
(c) adding labeled GDF-8 to the reaction vessel; and
(d) detecting labeled GDF-8 associated with the surface in the presence and absence of the biological sample.

55. The method of claim 54, wherein the biological sample is from a mammal, bird, reptile, or fish.

56. The method of claim 55, wherein the biological sample is from a mammal.

57. The method of claim 56, wherein the mammal is a human.

58. The method of claim 54, wherein the biological sample is chosen from serum, blood, plasma, biopsy sample, tissue sample, cell suspension, saliva, oral fluid, cerebrospinal fluid, amniotic fluid, milk, colostrum, mammary gland secretion, lymph, urine, sweat, lacrimal fluid, gastric fluid, synovial fluid, and mucus.

59. The method of claim 54, wherein the biological sample is chosen from serum, blood, and plasma.

60. The method of claim 54, wherein the label is chosen from an enzyme, an epitope tag, a radiolabel, biotin, a dye, a fluorescent tag label, and a luminescent label.

61. The method of claim 60, wherein the label is biotin.

62. The method of claim 61, further comprising adding an avidin-enzyme conjugate.

63. The method of claim 62, further comprising adding a substrate that changes color, luminescence, or fluorescence in the presence of the enzyme.

64. A method to assess an individual's immune response to a first GDF-8 inhibitor, the method comprising:
(a) contacting a first GDF-8 inhibitor with a surface of a reaction vessel;
(b) adding a biological sample from an individual to the reaction vessel;
(c) adding a labeled second GDF-8 inhibitor to the reaction vessel; and
(d) detecting a labeled second GDF-8 inhibitor/antibody complex associated with the surface,
wherein detection of labeled complex indicates an immune response to the first GDF-8 inhibitor.

65. A method to assess an individual's immune response to a first GDF-8 inhibitor, the method comprising:
(a) contacting a GDF-8 inhibitor with a surface of a reaction vessel;
(b) adding a biological sample from an individual to the reaction vessel;
(c) adding a labeled GDF-8 protein to the reaction vessel; and
(d) comparing the amount of labeled GDF-8 protein associated with the surface in the test sample to a control sample,
wherein detection of a decreased level of labeled complex indicates an immune response to the GDF-8 inhibitor.

66. A method to assess an individual's immune response to a first GDF-8 inhibitor, the method comprising:
(a) providing a host cell comprising a reporter gene construct in a reaction vessel, wherein the construct comprises a GDF-8-responsive control element and a reporter gene;
(b) adding an amount of mature GDF-8 protein to the vessel sufficient to activate expression of the reporter gene;
(c) adding an amount of MYO-029 to the vessel of step (b) sufficient to modulate the GDF-8 activation of the reporter gene;
(d) adding a biological sample to the reaction vessel of step (c); and
(e) detecting reporter gene expression in the presence and absence of the biological sample.


SEQUENCE LISTING (20 sequences)

SEQ ID NO: 1 - protein, 109 aa, Homo sapiens
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 2 - protein, 375 aa, Human
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 3 - DNA, 747 bp, Homo sapiens
caggtgcagctggtgcaatctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagacgagaactgggggttcgacccctggggccagggaaccctggtcaccgtctcgagtggaggcggcggttcaggcggaggtggctctggcggtggcggaagtgcactttcctatgagctgactcagccaccctcagtgtccgtgtctccaggacagacagccaccattacctgctctggacatgcactgggggacaaatttgtttcctggtatcagcagggatcaggccagtcccctgtattggtcatctatgacgatacccagcggccctcagggatccctgggcgattctctggctccaactctgggaacacagccactctgaccatcagcgggacccaggctatggatgaggctgactatttttgtcaggcgtgggacagcagcttcgtattcggcggagggaccaaggtcaccgtcctaggtgcggccgcacatcatcatcaccatcac

SEQ ID NO: 4 - protein, 249 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDENWGFDPWGQGTLVTVSSGGGGSGGGGSGGGGSALSYELTQPPSVSVSPGQTATITCSGHALGDKFVSWYQQGSGQSPVLVIYDDTQRPSGIPGRFSGSNSGNTATLTISGTQAMDEADYFCQAWDSSFVFGGGTKVTVLGAAAHHHHHH

SEQ ID NO: 5 - DNA, 351 bp, Homo sapiens
caggtgcagctggtgcaatctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagacgagaactgggggttcgacccctggggccagggaaccctggtcaccgtctcgagt

SEQ ID NO: 6 - protein, 117 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDENWGFDPWGQGTLVTVSS

SEQ ID NO: 7 - DNA, 315 bp, Homo sapiens
tcctatgagctgactcagccaccctcagtgtccgtgtctccaggacagacagccaccattacctgctctggacatgcactgggggacaaatttgtttcctggtatcagcagggatcaggccagtcccctgtattggtcatctatgacgatacccagcggccctcagggatccctgggcgattctctggctccaactctgggaacacagccactctgaccatcagcgggacccaggctatggatgaggctgactatttttgtcaggcgtgggacagcagcttcgtattcggcggagggaccaaggtcaccgtccta

SEQ ID NO: 8 - protein, 105 aa, Homo sapiens
SYELTQPPSVSVSPGQTATITCSGHALGDKFVSWYQQGSGQSPVLVIYDDTQRPSGIPGRFSGSNSGNTATLTISGTQAMDEADYFCQAWDSSFVFGGGTKVTVL

SEQ ID NO: 9 - DNA, 747 bp, Homo sapiens
caggtgcagctggtgcaatctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagacgagaactgggggttcgacccctggggccagggaaccctggtcaccgtctcgagtggaggcggcggttcaggcggaggtggctctggcggtggcggaagtgcactttcctatgagctgactcagccaccctcagtgtccgtgtctccaggacagacagccagcattacctgctctggacatgcactgggggacaaatttgtttcctggtatcagcagaagccaggccagtcccctgtattggtcatctatgacgatacccagcggccctcagggatccctgagcgattctctggctccaactctgggaacacagccactctgaccatcagcgggacccaggctatggatgaggctgactattactgtcaggcgtgggacagcagcttcgtattcggcggagggaccaaggtcaccgtcctaggtgcggccgcacatcaccatcaccatcac

SEQ ID NO: 10 - protein, 249 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDENWGFDPWGQGTLVTVSSGGGGSGGGGSGGGGSALSYELTQPPSVSVSPGQTASITCSGHALGDKFVSWYQQKPGQSPVLVIYDDTQRPSGIPERFSGSNSGNTATLTISGTQAMDEADYYCQAWDSSFVFGGGTKVTVLGAAAHHHHHH

SEQ ID NO: 11 - DNA, 351 bp, Homo sapiens; identical to SEQ ID NO: 5

SEQ ID NO: 12 - protein, 117 aa, Homo sapiens; identical to SEQ ID NO: 6

SEQ ID NO: 13 - DNA, 315 bp, Homo sapiens
tcctatgagctgactcagccaccctcagtgtccgtgtctccaggacagacagccagcattacctgctctggacatgcactgggggacaaatttgtttcctggtatcagcagaagccaggccagtcccctgtattggtcatctatgacgatacccagcggccctcagggatccctgagcgattctctggctccaactctgggaacacagccactctgaccatcagcgggacccaggctatggatgaggctgactattactgtcaggcgtgggacagcagcttcgtattcggcggagggaccaaggtcaccgtccta

SEQ ID NO: 14 - protein, 105 aa, Homo sapiens
SYELTQPPSVSVSPGQTASITCSGHALGDKFVSWYQQKPGQSPVLVIYDDTQRPSGIPERFSGSNSGNTATLTISGTQAMDEADYYCQAWDSSFVFGGGTKVTVL

SEQ ID NO: 15 - protein, 5 aa, Homo sapiens
SYYMH

SEQ ID NO: 16 - protein, 17 aa, Homo sapiens
IINPSGGSTSYAQKFQG

SEQ ID NO: 17 - protein, 8 aa, Homo sapiens
DENWGFDP

SEQ ID NO: 18 - protein, 11 aa, Homo sapiens
SGHALGDKFVS

SEQ ID NO: 19 - protein, 7 aa, Homo sapiens
DDTQRPS

SEQ ID NO: 20 - protein, 7 aa, Homo sapiens
QAWDSSF